(19) United States
(12) Patent Application Publication
(10) Pub. No.: US 2023/0293399 A1
(43) Pub. Date: Sep. 21, 2023

(54) SUNSCREEN-ACTIVE MIXTURES, METHODS OF FORMING TREATED MIXTURES FOR SUNSCREEN COMPOSITIONS, SUNSCREEN COMPOSITIONS, AND METHODS OF FORMING SUNSCREEN COMPOSITIONS

(71) Applicant: SAV Consultants LLC, Commack, NY (US)

(72) Inventors: Shetal Amit SHAH, Commack, NY (US); Tian WANG, Oviedo, FL (US); Michael INGRASSIA, Hauppauge, NY (US)

(21) Appl. No.: 18/184,503

(22) Filed: Mar. 15, 2023

Related U.S. Application Data
(60) Provisional application No. 63/320,622, filed on Mar. 16, 2022.

(51) Int. Cl. A61K 8/06; A61Q 17/04

(52) U.S. Cl. CPC A61K 8/068; A61Q 17/04; A61K 2800/412; A61K 2800/805

(57) ABSTRACT

Sunscreen-active mixtures, methods of forming treated mixtures for sunscreen compositions, sunscreen compositions, and methods of forming sunscreen compositions are disclosed herein. The sunscreen-active mixtures include an inorganic, sunscreen-active material, a first water-soluble polymer with a first glass transition temperature of at least 45° C., a second water-soluble polymer that has a second glass transition temperature that is less than the first glass transition temperature, and a surfactant. The sunscreen compositions include sunscreen-active material particles, which include the sunscreen-active mixture, and a cosmetic emulsion. An average particle size of the sunscreen-active material particles is at least 100 nm and at most 15 micrometers. The methods of forming the treated mixture include heating an untreated mixture and grinding the untreated mixture to generate the treated mixture. The methods of forming the sunscreen composition include providing sunscreen-active material particles, which include the sunscreen-active mixture, and dispersing the sunscreen-active material particles within a cosmetic emulsion.

### CROSS REFERENCES

The following applications and materials are incorporated herein, in their entireties, for all purposes: U.S. Provisional Patent Application No. 63/320,622, filed on Mar. 16, 2022.

### TECHNICAL FIELD

This disclosure relates generally to sunscreen-active mixtures, to methods of forming treated mixtures for sunscreen compositions, to sunscreen compositions, and to methods of forming sunscreen compositions.

### BACKGROUND

The sun emits radiation across the electromagnetic spectrum, including ultraviolet (UV) radiation. Prolonged skin exposure to the UV radiation damages skin cell DNA and can results in skin irritation and sunburns. Additionally, prolonged exposure to UV radiation prematurely ages the skin and increases the risk of several skin cancers and other skin disorders.

Sunscreens are topical products that can be applied to the skin to block, absorb, and/or scatter UV radiation before it can damage skin cells. While there are many commercially available sunscreens, the amount of UV protection provided by these commercially available sunscreens is limited. Thus, there exists a need for improved sunscreen-active mixtures, for improved methods of forming treated mixtures for sunscreen compositions, for improved sunscreen compositions, and for improved methods of forming sunscreen compositions.

### SUMMARY

Sunscreen-active mixtures, methods of forming treated mixtures for sunscreen compositions, sunscreen compositions, and methods of forming sunscreen compositions are disclosed herein. The sunscreen-active mixtures include an inorganic, sunscreen-active material, a first water-soluble polymer with a first glass transition temperature of at least 45° C., a second water-soluble polymer that has a second glass transition temperature that is less than the first glass transition temperature, and a surfactant.

The methods of forming the treated mixture include heating an untreated mixture and grinding the untreated mixture to generate the treated mixture. The untreated mixture includes the sunscreen-active mixture and the heating includes heating to a heated temperature that is within a threshold temperature differential of the first glass transition temperature of the first water-soluble polymer and that also is greater than the second glass transition temperature of the second water-soluble polymer.

The sunscreen compositions include sunscreen-active material particles, which include the sunscreen-active mixture, and a cosmetic emulsion. An average particle size of the inorganic, sunscreen-active material within the sunscreen-active material particles is at least 100 nm and at most 15 micrometers.

The methods of forming the sunscreen composition include providing sunscreen-active material particles, which include the sunscreen-active mixture. These methods also include dispersing the sunscreen-active material particles within a cosmetic emulsion.

### BRIEF DESCRIPTION OF THE DRAWINGS

FIG. 1 is a schematic illustration of examples of sunscreen-active material particles that may be included within sunscreen compositions, according to the present disclosure.

FIG. 2 is a schematic illustration of chips that may be utilized to form and/or to define sunscreen-active material particles, according to the present disclosure.

FIG. 3 is a schematic illustration of examples of sunscreen compositions that include sunscreen-active material particles, according to the present disclosure.

FIG. 4 is a flowchart illustrating examples of methods of manufacturing a treated mixture for a sunscreen composition, according to the present disclosure.

FIG. 5 is a flowchart illustrating examples of methods of forming a sunscreen composition, according to the present disclosure.

FIG. 6 is a table that describes components and relative compositional amounts for an example of sunscreen-active material particles, according to the present disclosure.

FIG. 7 is a table that describes components, relative compositional amounts, and achieved SPF for two sunscreen compositions, with Composition 1 being an example of a sunscreen composition according to the present discourse and Composition 2 being an example of a commercially available mineral sunscreen.

FIG. 8 is a table that describes components and relative compositional amounts for a hybrid sunscreen composition, according to the present disclosure.

FIG. 9 is a table that describes components and relative compositional amounts for a color sunscreen composition, according to the present disclosure.

FIG. 10 is a table that describes components and relative compositional amounts for a spray sunscreen composition, according to the present disclosure.

FIG. 11 is a table that describes components and relative compositional amounts for a cream sunscreen composition, according to the present disclosure.

FIG. 12 is a table that describes components and relative compositional amounts for a serum sunscreen composition, according to the present disclosure.

### DETAILED DESCRIPTION

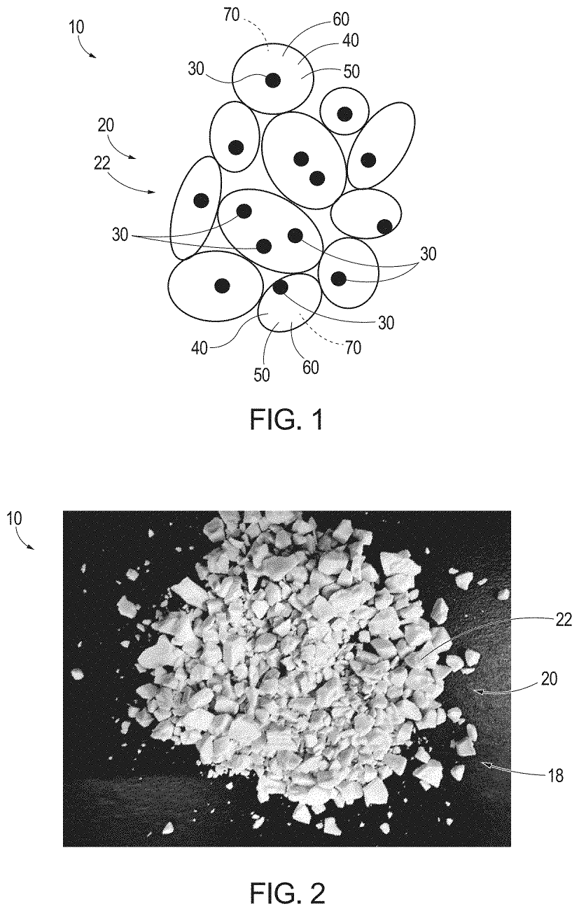

FIGS. 1-12 provide examples of sunscreen-active mixtures 22, of sunscreen-active material-particles 20 that include sunscreen-active mixtures 22, of sunscreen compositions 10 that include sunscreen-active material particles 20, of methods 200 of forming a treated mixture, and/or of methods 300 of forming a sunscreen composition, according to the present disclosure. Elements that serve a similar, or at least substantially similar, purpose are labeled with like numbers in each of FIGS. 1-12, and these elements may not be discussed in detail herein with reference to each of FIGS. 1-12. Similarly, all elements may not be labeled in each of FIGS. 1-12, but reference numerals associated therewith may be utilized herein for consistency. Elements, components, and/or features that are discussed herein with reference to one or more of FIGS. 1-12 may be included in and/or utilized with any of FIGS. 1-12 without departing from the scope of the present disclosure.

In general, elements that are likely to be included in a particular embodiment are illustrated in solid lines, while elements that are optional are illustrated in dashed lines. However, elements that are shown in solid lines may not be essential to all embodiments and, in some embodiments, may be omitted without departing from the scope of the present disclosure.

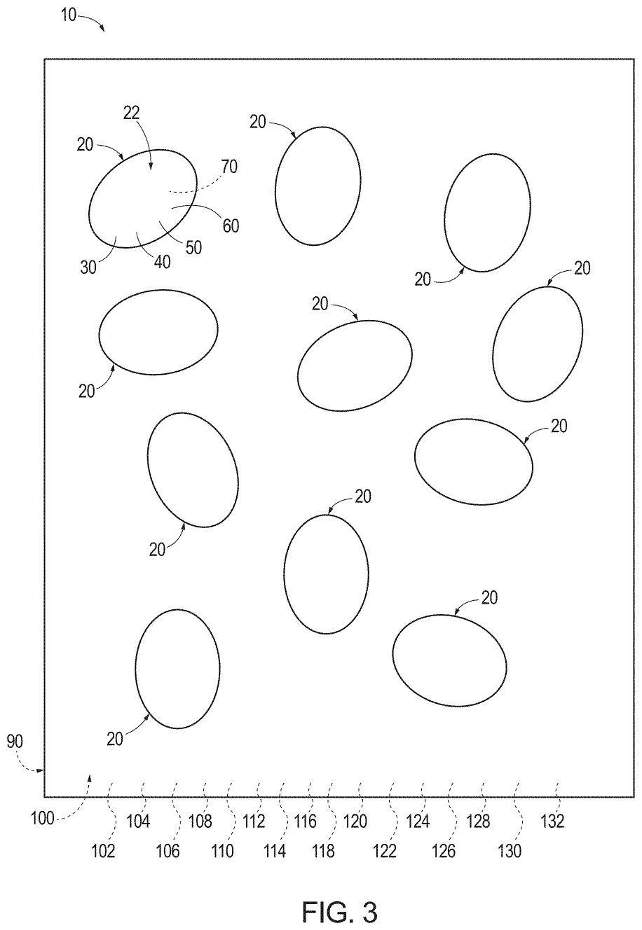

FIG. 1 is a schematic illustration of examples of sunscreen-active material particles 20 that may be included within sunscreen compositions 10, according to the present disclosure. FIG. 2 is a schematic illustration of chips 18 that may be utilized to form and/or to define sunscreen-active material particles 20, according to the present disclosure. FIG. 3 is a schematic illustration of examples of sunscreen compositions 10 that include sunscreen-active material particles 20, according to the present disclosure. Sunscreen-active material particles 20 also may be referred to herein as and/or may be water-dispersible, polymer-coated, inorganic, sunscreen-active material particles 20.

As illustrated in solid lines in FIGS. 1-2, chips 18 and/or sunscreen-active material particles 20 include a sunscreen-active mixture 22, which also may be referred to herein as a mixture 22. As perhaps best illustrated in FIG. 1, sunscreen-active mixture 22 includes an inorganic, sunscreen-active material 30, a first water-soluble polymer 40, a second water-soluble polymer 50, and a surfactant 60. As illustrated in dashed lines in FIG. 1, sunscreen-active mixture 22 also may include one or more additional mixture components 70, examples of which include iron oxide and/or water.

As illustrated in FIG. 3, sunscreen compositions 10 include sunscreen-active material particles 20. In addition, sunscreen compositions 10 may include one or more additional composition components 100, examples of which include an oil component 102, an aqueous component 104, an emulsifying component 106, a humectant component 108, a gel-forming component 110, a chemical sunscreen component 112, a colorant component 114, a preservative component 116, a moisturizing component 118, an anti-foaming component 120, an anti-inflammatory component 122, a neutralizing component 124, a thickening component 126, a filler component 128, a mineral sunscreen component 130, and/or a surfactant component 132. One or more of these components may be included in, may at least partially define, may be dispersed within, and/or may be suspended within a cosmetic emulsion 90. Sunscreen-active material particles 20, or individual sunscreen-active material particles 20, also may be dispersed and/or suspended within cosmetic emulsion 90. Stated differently, water-dispersible, polymer-coated, inorganic, sunscreen-active material particles 20 may be incorporated into and/or with cosmetic emulsion 90 and/or the other components, which are listed above, to form, to define, and/or for use as topical sunscreen products in the form of sunscreen compositions 10.

The effectiveness of a sunscreen in blocking UV radiation is measured in Sun Protection Factor (SPF). The SPF number increases by the amount of UV radiation it blocks, with SPF 50 blocking about 98% of UV radiation with wavelengths between 280 nm and 400 nm. A variety of sunscreen compositions are commercially available with different active ingredients that block UV radiation. Generally, sunscreens are classified into two categories: chemical sunscreens and mineral sunscreens.

Conventional chemical sunscreens use organic chemical compositions, such as oxybenzone, avobenzone, and octisalate as active ingredients to absorb UV radiation and convert it to heat. However, these compounds may irritate people with sensitive skin. Additionally, a small percentage of the active ingredients in chemical sunscreens may be absorbed into the body. Further, some chemical sunscreens may be harmful to the environment. Accordingly, conventional chemical sunscreens are banned or discouraged in several jurisdictions. As such, it may be advantageous that examples of sunscreen compositions 10, according to the present disclosure, may not include, or may not be required to include, such organic chemical compositions. However, sunscreen compositions 10 are not precluded from including such organic chemical compositions, as discussed in more detail herein.

Mineral sunscreens use particulates of inorganic metal oxides as active ingredients to block, scatter, and absorb UV radiation. Mineral sunscreens may have advantages over chemical sunscreens in that they may be safer for the environment, may cause less skin irritation, and/may not contain certain organic compounds that may be absorbed through the skin. Currently, Zinc Oxide (ZnO) and Titanium Dioxide (TiO₂) are approved in the United States and Europe for use as active ingredients in mineral sunscreens.

Current commercially available mineral sunscreens may have some disadvantages. As an example, commercially available mineral sunscreen-active ingredients are water-insoluble and tend to agglomerate in a mixture containing water, such as an oil/water emulsion like a cosmetic lotion. Because of this agglomeration, commercially available mineral sunscreens tend to provide an uneven coating on the skin, which may limit both the effectiveness and the maximum SPF achieved by such commercially available mineral sunscreens. In addition, the permissible percentage of ZnO or TiO₂ in sunscreen is capped at 25 weight percent (wt %) in the United States. Commercially available mineral sunscreens with 25 wt % ZnO only provide a UV protection of about 50 SPF, which may be due to the size, distribution, and/or solubility of the mineral particulates within the commercially available mineral sunscreens. However, the sunscreen-active mixtures, methods of forming treated mixtures for sunscreen compositions, sunscreen compositions, and/or methods of forming sunscreen compositions, which are disclosed herein, may include and/or be mineral sunscreens that provide more uniform coverage and/or increased SPF when compared to the commercially available mineral sunscreens.

Further, commercially available mineral sunscreens have properties disfavored by some consumers. The presence of ZnO or TiO₂ in commercially available mineral sunscreens tends to give commercially available mineral sunscreen lotions a thicker, stickier coating on the skin when compared to chemical sunscreens, and this thicker, stickier coating may be unacceptable to some consumers. In addition, commercially available mineral sunscreens tend to leave a white cast and/or streaks when applied to the skin, which may be aesthetically unpleasing when contrasted with many natural skin tones.

Furthermore, it may be desirable to regulate the particle size of the active ingredients in mineral sunscreens. As an example, the larger the particle size, the less efficiently the mineral sunscreen-active ingredient can coat the skin. In commercially available mineral sunscreens, large particle sizes (e.g., greater than 15 microns), may less efficiently cover the skin and require relatively higher concentrations to achieve an acceptable SPF. In addition, it may be desirable to avoid smaller particles (e.g., less than 100 nm), as some jurisdictions may require that these smaller particles be labeled as nanoparticles.

Sunscreen compositions 10 that includes sunscreen-active material particles 20 and/or sunscreen-active mixtures 22, according to the present disclosure, may provide the benefits of commercially available mineral sunscreens. However, sunscreen compositions 10 also may avoid many of the disadvantages of commercially available mineral sunscreens and/or may provide advantages over commercially available mineral sunscreens.

As an example, and as discussed in more detail herein, methods of the present disclosure provide a novel mechanism via which the size of inorganic, sunscreen-active material 30 within sunscreen-active material particles 20 readily may be controlled and/or regulated. This may permit and/or facilitate formulation of sunscreen compositions 10 with a desired particle size, or particle size range, and provide a mechanism via which overly large and/or small particle sizes may be avoided.

As another example, and as also discussed in more detail herein, formulation of sunscreen-active material particles 20, which include inorganic, sunscreen-active material 30, first water-soluble polymer 40, second water-soluble polymer 50, and surfactant 60, may cause inorganic, sunscreen-active material 30 to be at least partially, or even completely, coated, enclosed, and/or encapsulated by and/or within first water-soluble polymer 40, second water-soluble polymer 50, and/or surfactant 60. This may cause sunscreen-active material particles 20 to be partially soluble and/or dispersible in water, thereby decreasing a potential for separation of sunscreen-active material 30 from a remainder of sunscreen composition 10 before and/or after application to a user's skin and/or increasing a uniformity of application to the user's skin when compared to commercially available mineral sunscreens. This may decrease a potential for streaking, which is common with commercially available mineral sunscreens, and/or may increase the SPF provided by sunscreen compositions 10 according to the present discourse.

As another example, and as also discussed in more detail herein, first water-soluble polymer 40 may have and/or define a first transition temperature that is greater than a second glass transition temperature of second water-soluble polymer 50. The first glass transition temperature may be, or may be selected to be, greater than a skin temperature of a user who applies sunscreen compositions 10, while the second glass transition temperature may be, or may be selected to be, less than or approximately equal to the skin temperature of the user. Such a configuration may permit sunscreen-active material particles 20 to remain partially intact upon application to the user's skin while still providing a certain degree of flexibility and/or spreading of the sunscreen-active material particles on the user's skin. This may improve overall coverage, or uniformity of coverage, of the user's skin by sunscreen-active material particles 20.

The present disclosure describes sunscreen-active material particles 20 incorporated into and/or within sunscreen compositions 10. It is within the scope of the present disclosure that sunscreen-active material particles 20 additionally or alternatively may be incorporated into other products, such as cosmetics, which are configured to be applied to a user's skin and for which SPF properties may be desired. With this in mind, sunscreen compositions 10 also may be referred to herein as and/or may be cosmetic compositions 10, UV-blocking compositions 10, and/or skin-applied compositions 10.

As discussed, sunscreen-active material particles 20 include sunscreen-active mixture 22. As also discussed, sunscreen-active mixture 20 includes inorganic, sunscreen-active material 30, first water-soluble polymer 40, second water-soluble polymer 50, and surfactant 60; and also may include one or more additional mixture components 70.

Inorganic, sunscreen-active material 30, which also may be referred to herein as a sunscreen-active material 30, may include any suitable inorganic compound and/or compounds that may block UV radiation, that may absorb UV radiation, and/or that may decrease a potential for UV radiation to contact the user's skin. This may include inorganic compounds that absorb, scatter, and/or block electromagnetic radiation in a wavelength range of approximately 290 nanometers to 400 nanometers. Examples of inorganic, sunscreen-active materials 30 include zinc oxide (ZnO) and/or titanium dioxide (TiO₂).

Sunscreen-active mixture 22 may include any suitable amount, proportion, and/or percentage of inorganic, sunscreen-active material 30. As examples, sunscreen-active mixture 22 may include at least 1 weight percent (wt %), at least 2 wt %, at least 4 wt %, at least 6 wt %, at least 8 wt %, at least 10 wt %, at least 15 wt %, at least 20 wt %, at least 25 wt %, at least 30 wt %, at least 40 wt %, at least 45 wt %, at least 50 wt %, at least 55 wt %, at least 60 wt %, at least 65 wt %, at least 70 wt %, at least 75 wt %, at least 80 wt %, at most 90 wt %, at most 85 wt %, at most 80 wt %, at most 75 wt %, at most 70 wt %, at most 60 wt %, at most 50 wt %, at most 40 wt %, at most 30 wt %, at most 20 wt %, and/or at most 10 wt % of inorganic, sunscreen-active material 30. In a specific example, sunscreen-active mixture 22 may include approximately 70 wt % inorganic, sunscreen-active material 30.

As discussed, first water-soluble polymer 40 may have and/or define a first glass transition temperature, and second water-soluble polymer 50 may have and/or define a second glass transition temperature, which may be less than the first glass transition temperature. As examples, a difference between the first glass transition temperature and the second glass transition temperature may be at least 2° C., at least 4° C., at least 6° C., at least 8° C., at least 10° C., at least 12° C., at least 14° C., at least 16° C., at least 18° C., at least 20° C., at most 30° C., at most 28° C., at most 26° C., at most 24° C., at most 22° C., at most 20° C., at most 18° C., at most 16° C., at most 14° C., at most 12° C., and/or at most 10° C.

The first glass transition temperature may have any suitable magnitude. Examples of the first glass transition temperature include temperatures of at least 35° C., at least 40° C., at least 45° C., at least 50° C., at least 55° C., at least 60° C., at least 65° C., at least 70° C., at least 75° C., at least 80° C., at most 90° C., at most 85° C., at most 80° C., at most 75° C., at most 70° C., at most 65° C., at most 60° C., at most 55° C., at most 50° C., at most 45° C., and/or at most 40° C. In a specific example, the first glass transition temperature may be approximately 55° C.

Similarly, the second glass transition temperature may have nay suitable magnitude. Examples of the second glass transition temperature include temperatures of at least 25° C., at least 30° C., at least 35° C., at least 40° C., at least 45° C., at least 50° C., at least 55° C., at least 60° C., at least 65° C., at least 70° C., at most 80° C., at most 75° C., at most 70° C., at most 65° C., at most 60° C., at most 55° C., at most 50° C., at most 45° C., at most 40° C., at most 35° C., and/or at most 30° C. In a specific example, the second glass transition temperature may be approximately 38° C.

First water-soluble polymer 40 and second water-soluble polymer 50 may have and/or include any suitable chemical structure. As examples, the first water-soluble polymer and/or the second water-soluble polymer may include and/or be a polyester and a polyesteramide. As another example, the first water-soluble polymer and/or the second water-soluble polymer may include and/or be an aromatic sulfopolyester. As yet another example, the first water-soluble polymer and/or the second water-soluble polymer may include and/or be a polymeric resin. As another examples, the first water-soluble polymer and/or the second water-soluble polymer may have and/or define an inherent viscosity of at least 0.25 dL/g, which may permit and/or facilitate performing methods 200, which are discussed in more detail herein.

In some examples, first water-soluble polymer 40 and/or second water-soluble polymer 50 may be linear polymers. Examples of the polymers include the following groups, or the ester or esteramide products of reactants including the following groups: a difunctional dicarboxylic acid; a difunctional sulfomonomer with either a metallic sulfonate group or a nitrogen-containing non-metallic sulfonate group attached to an aromatic or cycloaliphatic nucleus wherein the functional groups are hydroxy, carboxyl or amino, or a mixture thereof; at least one difunctional reactant selected from a glycol, or a mixture of a glycol having two —NRH groups, the glycol containing two —CH₂—OH groups; a difunctional reactant selected from hydroxycarboxylic acids having one —C(R)₂—OH group, aminocarboxylic acids having one —NRH group, and amino alcohols having one —C(R)₂—OH group and one —NRH group, of mixtures thereof; wherein each R of is H or an alkyl group containing 1 to 4 carbon atoms. Additional examples of suitable polymers include polymers with one or more of the following components: a dicarboxylic acid component; a difunctional sulfomonomer component; and a glycol component, including those having an aromatic dicarboxylic acid moiety, an aliphatic or cycloaliphatic glycol residue, and on average, per molecule, between five and eight sodiosulfo (SO₃⁻Na⁺) substituents on the aromatic dicarboxylic acid moiety.

More specific examples of first water-soluble polymer 40 include AQ™ 55S, a polyester-5 with a glass transition temperature of, or of approximately, 51-55° C., a sulfopolyester with a glass transition temperature of, or of approximately, 51-55° C., and/or a copolymer of diethylene glycol, 1,4-cyclohexanedimethanol and simple esters of isophthalic acid and sulfoisophthalic acid with a glass transition temperature of, or of approximately, 51-55° C. More specific examples of the second water-soluble polymer include AQ™ 38S, a polyester-5 with a glass transition temperature of, or of approximately, 35-38° C., a sulfopolyester with a glass transition temperature of, or of approximately, 35-38° C., and a copolymer of diethylene glycol, 1,4-cyclohexanedimethanol and simple esters of isophthalic acid and sulfoisophthalic acid with a glass transition temperature of, or of approximately, approximately 35-38° C.

Sunscreen-active mixture 22 may include any suitable amount, proportion, and/or percentage of first water-soluble polymer 40 and/or of second water-soluble polymer 50. As examples, sunscreen-active mixture 22 may include at least 0.001 wt %, at least 0.01 wt %, at least 0.1 wt %, at least 1 wt %, at least 2 wt %, at least 4 wt %, at least 6 wt %, at least 8 wt %, at least 10 wt %, at least 15 wt %, at least 20 wt %, at least 25 wt %, at least 30 wt %, at least 35 wt %, at least 40 wt %, at least 45 wt %, at least 50 wt %, at most 60 wt %, at most 55 wt %, at most 50 wt %, at most 45 wt %, at most 40 wt %, at most 35 wt %, at most 30 wt %, at most 25 wt %, at most 20 wt %, at most 18 wt %, at most 16 wt %, at most 14 wt %, at most 12 wt %, and/or or at most 10 wt % of the first water-soluble polymer and/or of the second water-soluble polymer. In a specific example, sunscreen-active mixture 22 may include approximately 10 wt % of the first water-soluble polymer and/or of the second water-soluble polymer.

First water-soluble polymer 40 and second water-soluble polymer 50 may be included in sunscreen-active mixture 22 in any suitable proportion, or relative proportion. As examples, a ratio of a mass of first water-soluble polymer 40 to a mass of second water-soluble polymer 50, within the sunscreen-active mixture, may be, or be approximately, 2:3, 1:1, 3:2, or between 2:3 and 3:2.

Surfactant 60 may include any suitable surfactant that may permit and/or facilitate binding of first water-soluble polymer 40 and/or second water-soluble polymer 50 to inorganic, sunscreen-active material 30. This may include surfactants that are pharmaceutically and/or cosmetically acceptable and/or that are approved and/or permissible for utilization within topically applied lotions, creams, sprays, and/or compositions, including sunscreens. Examples of surfactant 60 include an anionic surfactant, a cationic surfactant, an amphoteric surfactant, an ethoxylated surfactant, a lecithin, soy lecithin, Finester 2009 (i.e., Polyglycerin-3 (and) Polyglyceryl-3 Lactate/Laurate), polyglyceryl-3 lactate/laurate, polyglyceryl-3 laurate, and polyglycerin-3.

Sunscreen-active mixture 22 may include any suitable amount, proportion, and/or percentage of surfactant 60. As examples, the sunscreen-active mixture may include at least 0.001 wt %, at least 0.01 wt %, at least 0.1 wt %, at least 1 wt %, at least 2 wt %, at least 4 wt %, at least 6 wt %, at least 8 wt %, at least 10 wt %, at least 15 wt %, at least 20 wt %, at least 25 wt %, at least 30 wt %, at most 40 wt %, at most 35 wt %, at most 30 wt %, at most 25 wt %, at most 20 wt %, at most 18 wt %, at most 16 wt %, at most 14 wt %, at most 12 wt %, and/or at most 10 wt % of the surfactant. In a specific example, sunscreen-active mixture 22 may include approximately 10 wt % of the surfactant.

Surfactant 60 may include and/or be a single surfactant, a single class of surfactant, and/or a single surfactant molecule. Alternatively, surfactant 60 may include a plurality of surfactants, such as a first surfactant and a second surfactant. Examples of the first surfactant include a lecithin and/or soy lecithin. Examples of the second surfactant include Finester 2009, polyglyceryl-3 lactate/laurate, polyglyceryl-3 laurate, and/or polyglycerin-3.

The first surfactant and the second surfactant, when present, may be included in sunscreen-active mixture 22 in any suitable proportion, or relative proportion. As examples, a ratio of a mass of the first surfactant to a mass of the second surfactant may be, or be approximately, 2:3, 1:1, 3:2, or between 2:3 and 3:2.

Additional mixture components 70, when present, may include any suitable pharmaceutically and/or cosmetically acceptable components. Stated differently, additional mixture components 70 may include any suitable compound that is approved and/or permissible for utilization within topically applied lotions, creams, sprays, and/or compositions, including sunscreens. As an example, additional mixture components 70 includes an iron oxide, such as may be utilized to provide a desired color and/or tint to sunscreen-active material particles 20 and/or to sunscreen compositions 10 that include sunscreen-active material particles 20. Another example of additional mixture components 70 includes water, such as may be incorporated into sunscreen-active material particles 20 to suppress dust during methods 200, as discussed in more detail herein.

Sunscreen-active mixture 22 may include any suitable amount, proportion, and/or percentage of additional mixture components 70, including iron oxide and/or water. As examples, sunscreen-active mixture 22 may include at least 0.01 wt %, at least 0.1 wt %, at least 1 wt %, at least 2 wt %, at least 4 wt %, at least 6 wt %, at least 8 wt %, at least 10 wt %, at least 15 wt %, at least 20 wt %, at least 25 wt %, at least 30 wt %, at least 35 wt %, at least 40 wt %, at most 50 wt %, at most 45 wt %, at most 40 wt %, at most 35 wt %, at most 30 wt %, at most 25 wt %, at most 20 wt %, at most 18 wt %, at most 16 wt %, at most 15 wt %, at most 14 wt %, at most 12 wt %, at most 10 wt %, at most 8 wt %, at most 6 wt %, at most 4 wt %, and/or at most 2 wt % of the additional mixture components and/or of each of the additional mixture components that are included therein. In a specific example, sunscreen-active mixture 22 may include approximately 5 wt % water.

In some examples, and as discussed in more detail herein with reference to methods 200, inorganic, sunscreen-active material 30 may form and/or define a particulate inorganic, sunscreen-active material that includes a plurality of material particles. In such examples, particulate inorganic, sunscreen-active material 30 may have and/or define an average particle size. Examples of the average particle size include an average maximum particle dimension, an average minimum particle dimension, and/or an average effective particle diameter. Another example of the average particle size includes a Feret diameter. The material particles may be embedded within a mixture of first water-soluble polymer 40, second water-soluble polymer 50, and surfactant 60 to form and/or to define the sunscreen-active material particles.

More specific examples of the average particle size include particle sizes of at least 50 nanometers (nm), at least 100 nm, at least 200 nm, at least 300 nm, at least 400 nm, at least 500 nm, at least 600 nm, at least 700 nm, at least 800 nm, at least 900 nm, at least 1 micrometer, at least 2 micrometers, at least 4 micrometers, at least 6 micrometers, at least 8 micrometers, at least 10 micrometers, at most 20 micrometers, at most 18 micrometers, at most 16 micrometers, at most 15 micrometers, at most 14 micrometers, at most 12 micrometers, at most 10 micrometers, at most 8 micrometers, and/or at most 6 micrometers. A more specific example of the average particle size includes particles of at least 100 nm and at most 15 micrometers.

As discussed, sunscreen compositions 10, according to the present disclosure, include sunscreen-active material particles 20, which are discussed in more detail herein. Sunscreen compositions 10 also may include one or more additional composition components 100, examples of which are disclosed herein. As also discussed, the one or more additional composition components may at least partially define, may be dispersed within, and/or may be suspended within cosmetic emulsion 90. It is within the scope of the present disclosure that any of the additional composition components, which are disclosed herein, may at least partially define, may be referred to herein as at least partially defining, may be included in, and/or may be dispersed within cosmetic emulsion 90. With this in mind, cosmetic emulsion 90 also may be referred to herein as and/or may be an additional component mixture 100.

In some examples, sunscreen-active material particles 20 may be dispersed and/or suspended within cosmetic emulsion 90. Stated differently, sunscreen-active material particles 20 may include and/or be a plurality of distinct, individual, and/or spaced-apart sunscreen-active material particles 20 that may be dispersed and/or suspended within the cosmetic emulsion.

Sunscreen composition 10 may include any suitable amount, proportion, and/or percentage of sunscreen-active material particles 20. As examples, sunscreen composition 10 may include at least 0.001 wt %, at least 0.01 wt %, at least 0.1 wt %, at least 1 wt %, at least 2 wt %, at least 3 wt %, at least 4 wt %, at least 5 wt %, at least 6 wt %, at least 8 wt %, at least 10 wt %, at least 15 wt %, at least 20 wt %, at least 25 wt %, at least 30 wt %, at least 35 wt %, at least 40 wt %, at most 50 wt %, at most 45 wt %, at most 40 wt %, at most 35 wt %, at most 30 wt %, at most 25 wt %, at most 20 wt %, at most 15 wt % and/or at most 10 wt % of the sunscreen-active material particles.

Cosmetic emulsion 90, when present, may include and/or be any pharmaceutically and/or cosmetically acceptable material and/or materials. Stated differently, cosmetic emulsion 90 may include any suitable liquid, or at least partially liquid, mixture that is approved and/or permissible for utilization within topically applied lotions, creams, sprays, and/or compositions, including sunscreens. Examples of cosmetic emulsion 90 include any suitable combination of additional composition components 100, which are disclosed herein. In a specific example, cosmetic emulsion 90 includes, or includes at least, oil component 102, aqueous component 104, and emulsifying component 106.

When sunscreen composition 10 includes cosmetic emulsion 90, the cosmetic emulsion may include any suitable amount, proportion, and/or percentage of the cosmetic emulsion. As examples, the sunscreen composition may include at least 50 wt %, at least 55 wt %, at least 60 wt %, at least 65 wt %, at least 70 wt %, at least 80 wt %, at least 85 wt %, at least 90 wt %, at most 99.9 wt %, at most 99 wt %, at most 95 wt %, at most 90 wt %, at most 85 wt %, at most 80 wt %, at most 75 wt %, at most 70 wt %, at most 65 wt %, and/or at most 60 wt % of the cosmetic emulsion.

Oil component 102, when present, may include any suitable material and/or liquid that is insoluble, or at least substantially insoluble, in water. This may include oil components 102 that are pharmaceutically and/or cosmetically acceptable and/or that are approved and/or permissible for utilization within topically applied lotions, creams, sprays, and/or compositions, including sunscreens. Examples of oil component 102 include a natural oil, a vegetable oil, coconut oil, a hydrocarbon, a hydrocarbon oil, a mineral oil, hydrogenated polyisobutene, a fatty alcohol, octyldodecanol, an ester, a C12-C15 alkyl benzoate, a C12 alkyl benzoate, a C13 alkyl benzoate, a C14 alkyl benzoate, a C15 alkyl benzoate, a diester, propylene glycol dipelargonate, a triester, glyceryl trioctanoate, a sterol derivative, lanolin, a wax, an animal waxes, beeswax, a plant wax, carnauba wax, a mineral wax, ozokerite, a petroleum wax, paraffin wax, a synthetic wax, polyethylene, a silicone, a volatile silicone, cyclomethicone, a polymeric silicone, dimethicone, an alkylated derivatives of polymeric silicones, cetyl dimethicone, lauryl trimethicone, a hydroxylated derivatives of polymeric silicones, dimethiconol, cetearyl alcohol, dimethicone/vinyl dimethicone crosspolymer, coco-caprylate/caprate, isohexadecane, C18-36 acid triglyceride, isododecane, butylated hydroxy-toluene, and isostearic acid.

When sunscreen composition 10 includes oil component 102, the sunscreen composition may include any suitable amount, proportion, and/or percentage of the oil component. As examples, the sunscreen composition may include at least 0.001 wt %, at least 0.01 wt %, at least 0.1 wt %, at least 1 wt %, at least 2 wt %, at least 3 wt %, at least 4 wt %, at least 5 wt %, at least 6 wt %, at least 8 wt %, at least 10 wt %, at least 15 wt %, at least 20 wt %, at least 25 wt %, at least, at least 30 wt %, at most 40 wt %, at most 35 wt %, at most 30 wt %, at most 25 wt %, at most 20 wt %, at most 15 wt %, and/or at most 10 wt % of the oil component.

Aqueous component 104, when present, may include any suitable material and/or liquid that includes water, that is miscible, or at least substantially miscible, in water, that consists essentially of water, and/or that includes water as a primary, or as a majority, component. This may include aqueous components 104 that are pharmaceutically and/or cosmetically acceptable and/or that are approved and/or permissible for utilization within topically applied lotions, creams, sprays, and/or compositions, including sunscreens.

When sunscreen composition 10 includes aqueous component 104, the sunscreen composition may include any suitable amount, proportion, and/or percentage of the aqueous component. As examples, the sunscreen composition may include at least 1 wt %, at least 2 wt %, at least 3 wt %, at least 4 wt %, at least 5 wt %, at least 6 wt %, at least 8 wt %, at least 10 wt %, at least 15 wt %, at least 20 wt %, at least 25 wt %, at least, at least 30 wt %, at least 35 wt %, at least 40 wt %, at least 45 wt %, at least 50 wt %, at most 80 wt %, at most 70 wt %, at most 60 wt %, at most 50 wt %, at most 40 wt %, at most 30 wt %, at most 20 wt %, at most 10 wt %, and/or at most 5 wt % of the aqueous component.

Emulsifying component 106, when present, may include any suitable material that stabilizes a two-phase liquid mixture, such as a mixture of oil component 102 and aqueous component 104. This may include emulsifying components 106 that are pharmaceutically and/or cosmetically acceptable and/or that are approved and/or permissible for utilization within topically applied lotions, creams, sprays, and/or compositions, including sunscreens. Examples of the emulsifying component include an oil-in-water emulsifier, an emulsifier having a hydrophilic lipophilic balance (HLB) of at least 6, a sorbitol derivative, sorbitan monolaurate, polysorbate 20, an ethoxylated alcohol, laureth-23, an ethoxylated fatty acid, PEG-1000 stearate, an amidoamine derivative, stearamidoethyl diethylamine, a sulfate ester, sodium lauryl sulfate, a phosphate ester, DEA cetyl phosphate, a fatty acid amine salt, TEA stearate, a water-in-oil emulsifier, an emulsifier having a hydrophilic lipophilic balance of 2 to 6, a sorbitan derivative, sorbitan laurate, sorbitan palmitate, an alkoxylated alcohol, laureth-4, a hydroxylated derivative of polymeric silicones, dimethicone copolyol, an alkylated derivatives of hydroxylated polymeric silicones, cetyl dimethicone copolyol, a glyceryl ester, polyglyceryl-4 isostearate, a beeswax derivative, sodium isostearoyl-2 lactylate, lecithin, glycerol monostearate, cetyl PEG/PPG-10/1 dimethicone, PEG 100 Stearate, sodium stearoyl glutamate, polyglyceryl-6 polyhydroxystearate, polyglyceryl-6 polyricinoleate, PEG-10 Dimethicone, and/or stearic acid.

When sunscreen composition 10 includes emulsifying component 106, the sunscreen composition may include any suitable amount, proportion, and/or percentage of the emulsifying component. As examples, the sunscreen composition may include at least 0.001 wt %, at least 0.01 wt %, at least 0.1 wt %, at least 1 wt %, at least 2 wt %, at least 3 wt %, at least 4 wt %, at least 5 wt %, at least 6 wt %, at least 8 wt %, at least 10 wt %, at least 15 wt %, at least 20 wt %, at most 30 wt %, at most 25 wt %, at most 20 wt %, at most 18 wt %, at most 16 wt %, at most 14 wt %, at most 12 wt %, at most 10 wt %, at most 8 wt %, at most 6 wt %, at most 4 wt %, at most 2 wt %, and/or at most 1 wt % of the emulsifying component.

Humectant component 108, when present, may include any suitable material that, upon application to the skin of the user, reduces moisture loss from this skin. This may include humectant components 108 that are pharmaceutically and/or cosmetically acceptable and/or that are approved and/or permissible for utilization within topically applied lotions, creams, sprays, and/or compositions, including sunscreens. Examples of the humectant component include propanediol and/or butylene glycol.

When sunscreen composition 10 includes humectant component 108, the sunscreen composition may include any suitable amount, proportion, and/or percentage of the humectant component. As examples, the sunscreen composition may include at least 0.01 wt %, at least 0.1 wt %, at least 1 wt %, at least 2 wt %, at least 3 wt %, at least 4 wt %, at least 5 wt %, at least 6 wt %, at least 8 wt %, at least 10 wt %, at least 15 wt %, at least 20 wt %, at most 30 wt %, at most 25 wt %, at most 20 wt %, at most 18 wt %, at most 16 wt %, at most 14 wt %, at most 12 wt %, at most 10 wt %, at most 8 wt %, at most 6 wt %, at most 4 wt %, at most 2 wt %, and/or at most 1 wt % of the humectant component.

Gel-forming component 110, when present, may include any suitable material that gels within sunscreen compositions 10 and/or that causes sunscreen compositions 10 to gel, or to at least partially gel. This may include gel-forming components 110 that are pharmaceutically and/or cosmetically acceptable and/or that are approved and/or permissible for utilization within topically applied lotions, creams, sprays, and/or compositions, including sunscreens. Examples of the gel-forming component include a hydrocollide and/or xanthan gum.

When sunscreen composition 10 includes gel-forming component 110, the sunscreen composition may include any suitable amount, proportion, and/or percentage of the gel-forming component. As examples, the sunscreen composition may include at least 0.001 wt %, at least 0.01 wt %, at least 0.1 wt %, at least 1 wt %, at least 2 wt %, at least 3 wt %, at least 4 wt %, at most 6 wt %, at most 5 wt %, at most 4 wt %, at most 3 wt %, at most 2 wt %, and/or at most 1 wt % of the gel-forming component.

Chemical sunscreen component 112, when present, may include any suitable chemical compound that may block UV radiation, that may absorb UV radiation, and/or that may decrease a potential for UV radiation to contact the user's skin. This may include chemical sunscreen components 112 that are pharmaceutically and/or cosmetically acceptable and/or that are approved and/or permissible for utilization within topically applied lotions, creams, sprays, and/or compositions, including sunscreens. Examples of the chemical sunscreen component include ethylhexyl salicylate, octocrylene, homosalate, and/or avobenzone.

When sunscreen composition 10 includes chemical sunscreen component 112, the sunscreen composition may include any suitable amount, proportion, and/or percentage of the chemical sunscreen component. As examples, the sunscreen composition may include at least 0.001 wt %, at least 0.01 wt %, at least 0.1 wt %, at least 1 wt %, at least 2 wt %, at least 3 wt %, at least 4 wt %, at least 5 wt %, at least 6 wt %, at least 8 wt %, at least 10 wt %, at least 15 wt %, at least 20 wt %, at most 30 wt %, at most 25 wt %, at most 20 wt %, at most 18 wt %, at most 16 wt %, at most 14 wt %, at most 12 wt %, at most 10 wt %, at most 8 wt %, at most 6 wt %, at most 4 wt %, at most 2 wt %, and/or at most 1 wt % of the chemical sunscreen component.

Colorant component 114, when present, may include any suitable material that may provide a desired color, tint, and/or hue to sunscreen compositions 10 before and/or after application of the sunscreen compositions to the user's skin. This may include colorant components 114 that are pharmaceutically and/or cosmetically acceptable and/or that are approved and/or permissible for utilization within topically applied lotions, creams, sprays, and/or compositions, including sunscreens. Examples of the colorant component include titanium dioxide, red iron oxide, yellow iron oxide, black iron oxide, an iron oxide, an FD&C dye, lake, and iron blue.

When sunscreen composition 10 includes colorant component 114, the sunscreen composition may include any suitable amount, proportion, and/or percentage of the colorant component. As examples, the sunscreen composition may include at least 0.001 wt %, at least 0.01 wt %, at least 0.1 wt %, at least 1 wt %, at least 2 wt %, at least 3 wt %, at least 4 wt %, at least 5 wt %, at least 6 wt %, at least 8 wt %, at least 10 wt %, at least 15 wt %, at least 20 wt %, at least 30 wt %, at least 40 wt % at most 60 wt %, at most 55 wt %, at most 50 wt %, at most 45 wt %, at most 40 wt %, at most 35 wt %, at most 30 wt %, at most 25 wt %, at most 20 wt %, at most 18 wt %, at most 16 wt %, at most 14 wt %, at most 12 wt %, at most 10 wt %, at most 8 wt %, at most 6 wt %, at most 4 wt %, at most 2 wt %, and/or at most 1 wt % of the colorant component.

Preservative component 116, when present, may include any suitable material that may preserve and/or that may increase a shelf life of sunscreen compositions 10. This may include preservative components 116 that are pharmaceutically and/or cosmetically acceptable and/or that are approved and/or permissible for utilization within topically applied lotions, creams, sprays, and/or compositions, including sunscreens. An example of the preservative component includes caprylyl glycol.

When sunscreen composition 10 includes preservative component 116, the sunscreen composition may include any suitable amount, proportion, and/or percentage of the preservative component. As examples, the sunscreen composition may include at least 0.001 wt %, at least 0.01 wt %, at least 0.1 wt %, at least 0.2 wt %, at least 0.3 wt %, at least 0.4 wt %, at least 0.5 wt %, at least 0.6 wt %, at least 0.7 wt %, at least 0.8 wt %, at least 0.9 wt %, at least 1 wt %, at most 2 wt %, at most 1.8 wt %, at most 1.6 wt %, at most 1.4 wt %, at most 1.2 wt %, at most 1 wt %, at most 0.9 wt %, at most 0.8 wt %, at most 0.7 wt %, at most 0.6 wt %, at most 0.5 wt %, at most 0.4 wt %, at most 0.3 wt %, and/or at most 0.2 wt % of the preservative component.

Moisturizing component 118, when present, may include any suitable material that, subsequent to application to the user's skin, may moisturize, or increase a moisture content of, the user's skin. This may include moisturizing components 118 that are pharmaceutically and/or cosmetically acceptable and/or that are approved and/or permissible for utilization within topically applied lotions, creams, sprays, and/or compositions, including sunscreens. Examples of the moisturizing component include sodium hyaluronate, sucrose, and D-Panthenol.

When sunscreen composition 10 includes moisturizing component 118, the sunscreen composition may include any suitable amount, proportion, and/or percentage of the moisturizing component. As examples, the sunscreen composition may include at least 0.01 wt %, at least 0.05 wt %, at least 0.1 wt %, at least 0.2 wt %, at least 0.3 wt %, at least 0.4 wt %, at least 0.5 wt %, at most 2 wt %, at most 1.8 wt %, at most 1.6 wt %, at most 1.4 wt %, at most 1.2 wt %, at most 1 wt %, at most 0.9 wt %, at most 0.8 wt %, at most 0.7 wt %, at most 0.6 wt %, at most 0.5 wt %, at most 0.4 wt %, at most 0.3 wt %, and/or at most 0.2 wt % of the moisturizing component.

Anti-foaming component 120, when present, may include any suitable material and/or liquid that may decrease a potential for foaming of sunscreen compositions 10 during formulation thereof and/or during application to the user's skin. This may include anti-foaming components 120 that are pharmaceutically and/or cosmetically acceptable and/or that are approved and/or permissible for utilization within topically applied lotions, creams, sprays, and/or compositions, including sunscreens. An example of the anti-foaming component includes simethicone.

When sunscreen composition 10 includes anti-foaming component 120, the sunscreen composition may include any suitable amount, proportion, and/or percentage of the anti-foaming component. As examples, the sunscreen composition may include at least 0.01 wt %, at least 0.05 wt %, at least 0.1 wt %, at least 0.2 wt %, at least 0.3 wt %, at least 0.4 wt %, at least 0.5 wt %, at most 1 wt %, at most 0.9 wt %, at most 0.8 wt %, at most 0.7 wt %, at most 0.6 wt %, at most 0.5 wt %, at most 0.4 wt %, at most 0.3 wt %, at most 0.2 wt %, and/or at most 0.1 wt % of the anti-foaming component.

Anti-inflammatory component 122, when present, may include any suitable material that, upon application to the user's skin, may decrease inflammation, or perceived inflammation. This may include anti-inflammatory components 122 that are pharmaceutically and/or cosmetically acceptable and/or that are approved and/or permissible for utilization within topically applied lotions, creams, sprays, and/or compositions, including sunscreens. An example of the anti-inflammatory component includes caffeine.

When sunscreen composition 10 includes anti-inflammatory component 122, the sunscreen composition may include any suitable amount, proportion, and/or percentage of the anti-inflammatory component. As examples, the sunscreen composition may include at least 0.01 wt %, at least 0.05 wt %, at least 0.1 wt %, at least 0.2 wt %, at least 0.3 wt %, at least 0.4 wt %, at least 0.5 wt %, at most 1 wt %, at most 0.9 wt %, at most 0.8 wt %, at most 0.7 wt %, at most 0.6 wt %, at most 0.5 wt %, at most 0.4 wt %, at most 0.3 wt %, at most 0.2 wt %, and/or at most 0.1 wt % of the anti-inflammatory component.

Neutralizing component 124, when present, may include any suitable material that, upon application to the user's skin, may neutralize a pH of the user's skin. This may include neutralizing components 124 that are pharmaceutically and/or cosmetically acceptable and/or that are approved and/or permissible for utilization within topically applied lotions, creams, sprays, and/or compositions, including sunscreens. Examples of the neutralizing component include caprylyl glycol and/or tromethamine.

When sunscreen composition 10 includes neutralizing component 124, the sunscreen composition may include any suitable amount, proportion, and/or percentage of the neutralizing component. As examples, the sunscreen composition may include at least 0.01 wt %, at least 0.05 wt %, at least 0.1 wt %, at least 0.2 wt %, at least 0.4 wt %, at least 0.6 wt %, at least 0.8 wt %, at least 1 wt %, at least 2 wt %, at least 3 wt %, at least 4 wt %, at most 6 wt %, at most 5 wt %, at most 4 wt %, at most 3 wt %, at most 2 wt %, at most 1 wt %, at most 0.8 wt %, at most 0.6 wt %, at most 0.4 wt %, at most 0.2 wt %, and/or at most 0.1 wt % of the neutralizing component.

Thickening component 126, when present, may include any suitable material and/or liquid that may thicken, or increase a viscosity, of sunscreen compositions 10. This may include thickening components 126 that are pharmaceutically and/or cosmetically acceptable and/or that are approved and/or permissible for utilization within topically applied lotions, creams, sprays, and/or compositions, including sunscreens. An example of the thickening component includes disteardimonium hectorite. An example of an activator for the thickening component includes propylene carbonate.

When sunscreen composition 10 includes thickening component 126, the sunscreen composition may include any suitable amount, proportion, and/or percentage of the thickening component. As examples, the sunscreen composition may include at least 0.001 wt %, at least 0.01 wt %, at least 0.05 wt %, at least 0.1 wt %, at least 0.15 wt %, at least 0.2 wt %, at most 0.5 wt %, at most 0.4 wt %, at most 0.3 wt %, at most 0.2 wt %, and/or at most 0.1 wt % of the thickening component.

Filler component 128, when present, may include any suitable material that may impart shimmer and/or sparkle to sunscreen compositions 10 and/or that may add body to the sunscreen compositions. This may include filler components 128 that are pharmaceutically and/or cosmetically acceptable and/or that are approved and/or permissible for utilization within topically applied lotions, creams, sprays, and/or compositions, including sunscreens. An example of the filler component includes mica.

When sunscreen composition 10 includes filler component 128, the sunscreen composition may include any suitable amount, proportion, and/or percentage of the filler component. As examples, the sunscreen composition may include at least 0.01 wt %, at least 0.05 wt %, at least 0.1 wt %, at least 0.2 wt %, at least 0.4 wt %, at least 0.6 wt %, at least 0.8 wt %, at least 1 wt %, at least 2 wt %, at least 3 wt %, at least 4 wt %, at most 10 wt %, at most 8 wt %, at most 6 wt %, at most 5 wt %, at most 4 wt %, at most 3 wt %, at most 2 wt %, at most 1 wt %, at most 0.8 wt %, at most 0.6 wt %, at most 0.4 wt %, at most 0.2 wt %, and/or at most 0.1 wt % of the filler component.

Mineral sunscreen component 130, when present, may include any suitable material that may block UV radiation, that may absorb UV radiation, and/or that may decrease a potential for UV radiation to contact the user's skin. This may include mineral sunscreen components 130 that are pharmaceutically and/or cosmetically acceptable and/or that are approved and/or permissible for utilization within topically applied lotions, creams, sprays, and/or compositions, including sunscreens. Examples of the mineral sunscreen component are disclosed herein with reference to inorganic, sunscreen-active material 30. The inclusion of both sunscreen-active material particles 20 and mineral sunscreen component 130, within sunscreen compositions 10, further may increase an SPF of the sunscreen compositions, such as via permitting mineral sunscreen component 130, which may be a particulate mineral sunscreen component 130, to fill in and/or cover gaps and/or spaces between adjacent sunscreen-active material particles 20 on the user's skin.

When sunscreen composition 10 includes mineral sunscreen component 130, the sunscreen composition may include any suitable amount, proportion, and/or percentage of the mineral sunscreen component. As examples, the sunscreen composition may include at least 0.001 wt %, at least 0.01 wt %, at least 0.1 wt %, at least 1 wt %, at least 2 wt %, at least 4 wt %, at least 6 wt %, at least 8 wt %, at least 10 wt %, at least 15 wt %, at least 20 wt %, at most 30 wt %, at most 25 wt %, at most 20 wt %, at most 15 wt %, at most 10 wt %, at most 8 wt %, at most 6 wt %, at most 4 wt %, and/or at most 2 wt % of the mineral sunscreen component.

Surfactant component 122, when present, may include any suitable material that may function as a surfactant within sunscreen compositions 10. This may include surfactant components 132 that are pharmaceutically and/or cosmetically acceptable and/or that are approved and/or permissible for utilization within topically applied lotions, creams, sprays, and/or compositions, including sunscreens. Examples of the surfactant component are disclosed herein with reference to surfactant 60.

When sunscreen composition 10 includes surfactant component 132, the sunscreen composition may include any suitable amount, proportion, and/or percentage of the surfactant component. As examples, the sunscreen composition may include at least 0.001 wt %, at least 0.01 wt %, at least 0.05 wt %, at least 0.1 wt %, at least 0.2 wt %, at least 0.4 wt %, at least 0.6 wt %, at least 0.8 wt %, at least 1 wt %, at most 3 wt %, at most 2.5 wt %, at most 2 wt %, at most 1.5 wt %, at most 1 wt %, at most 0.8 wt %, at most 0.6 wt %, at most 0.4 wt %, at most 0.2 wt %, and/or at most 0.1 wt % of the surfactant component.

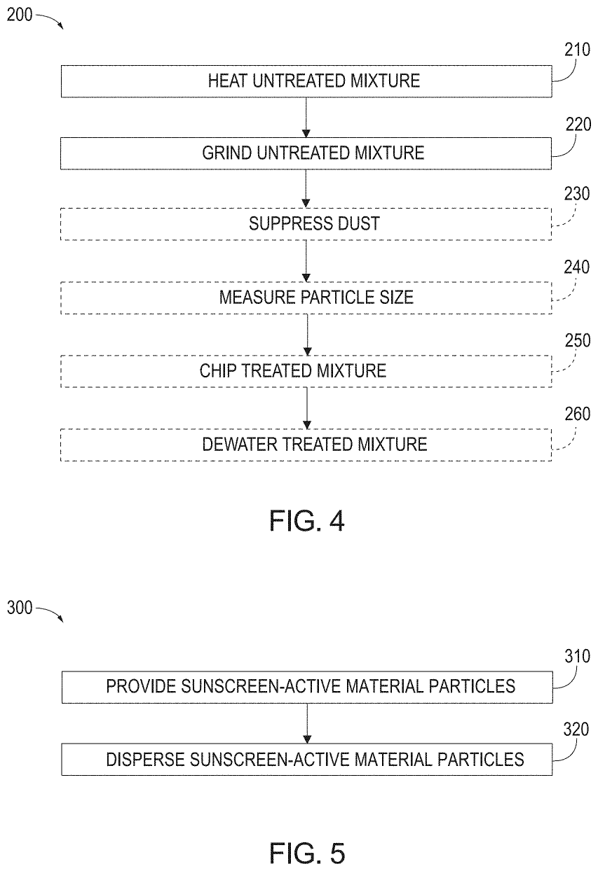

FIG. 4 is a flowchart illustrating examples of methods 200 of manufacturing a treated mixture for a sunscreen composition, according to the present disclosure. The treated mixture may be included in sunscreen compositions 10 as sunscreen-active material particles 20. Methods 200 include heating an untreated mixture at 210 and grinding the untreated mixture at 220. Methods 200 also may include suppressing dust at 230, measuring a particle size at 240, chipping the treated mixture at 250, and/or dewatering the treated mixture at 260.

Heating an untreated mixture at 210 may include heating the untreated mixture to a heated temperature. The untreated mixture may include and/or be any suitable sunscreen-active mixture 22 and/or component thereof, which are disclosed herein. As an example, the untreated mixture may include inorganic, sunscreen-active material 30, first water-soluble polymer 40, second water-soluble polymer 50, and surfactant 60. In such an example, the heated temperature may be within a threshold temperature differential of the first glass transition temperature of the first water-soluble polymer. In some examples, and prior to the heating at 210, methods 200 may include mixing and/or combining the inorganic, sunscreen-active material, the first water-soluble polymer, the second water-soluble polymer, the surfactant, and/or one or more additional components to form and/or to define the untreated mixture.

The heating at 210 may be performed with any suitable timing and/or sequence during methods 200. As examples, the heating at 210 may be performed at least partially, or even completely, concurrently with the grinding at 220, the suppressing at 230, the measuring at 240, the chipping at 250, and/or the dewatering at 260. As additional examples, the heating at 210 may be performed at least partially, or even completely, prior to the grinding at 220, the suppressing at 230, the measuring at 240, the chipping at 250, and/or the dewatering at 260. In some examples, the heating at 210 may include maintaining the untreated mixture at the heated temperature during the grinding at 210.

The threshold temperature differential may include and/or be any suitable temperature differential. Examples of the threshold temperature differential include at most 0.5° C., at most 1° C., at most 1.5° C., at most 2° C., at most 3° C., at most 4° C., at most 5° C., at most 6° C., at most 7° C., at most 8° C., at most 9° C., or at most 10° C. In some examples, the heated temperature may be greater than the second glass transition temperature of the second water-soluble polymer. In some examples, the heated temperature may be at most, or below, the first glass transition temperature. In some such examples, the heating at 210 may include maintaining the untreated mixture at, or below, the first glass transition temperature during the grinding at 220.

Grinding the untreated mixture at 220 may include grinding the untreated mixture to produce and/or to generate the treated mixture. In some examples, a chemical composition of the treated mixture may be at least substantially similar, or even identical, to a chemical composition of the untreated mixture. However, methods 200 may cause the inorganic, sunscreen-active material to be at least partially surrounded by, to be dispersed within, and/or to be at least partially encapsulated within a mixture of the first water-soluble polymer, the second water-soluble polymer, and the surfactant. This may increase an effectiveness of the sunscreen-active mixture when incorporated into sunscreen compositions 10 in the form of the treated mixture and/or as sunscreen-active material particles 20. Surprisingly, combining the first water-soluble polymer, the second water-soluble polymer, and the surfactant at, or near, the first glass transition temperature does not result in the second water-soluble polymer (with a lower second glass transition temperature) becoming too soft to be effectively ground into the treated mixture.

The grinding at 220 may be accomplished in any suitable manner that combines, mixes, compresses, shears, and/or breaks apart the untreated mixture to produce and/or generate the treated mixture. As an example, the grinding at 220 may include grinding with, via, utilizing, and/or on a roller mill that includes at least two rollers. In such an example, a first roller of the at least two rollers may be unheated, and a second roller of the at least two rollers may heated to a roller temperature that is within the threshold temperature differential. Stated differently, and in such an example, the heating at 210 may be at least partially, or even completely, performed via heating the second roller.

As another example, the grinding at 220 may include passing the untreated mixture through the roller mill and subsequently grinding the untreated mixture on the roller mill. In such a configuration, the passing may mix, or may further mix, components of the untreated mixture, while the grinding may decrease the average particle size of the inorganic, sunscreen-active material within the inorganic, sunscreen-active material particles of the treated mixture. The passing may be performed with rollers of the roller mill at a first spacing, and the grinding may be performed with the rollers of the roller mill at a second spacing, which is less than the first spacing.

As another example, the grinding at 220 may include extruding the untreated mixture with, via, and/or utilizing an extrusion device, or an extruder. In such an example, the extrusion device may an extrusion device heater, and the heating at 210 may include heating the untreated mixture with, via, and/or utilizing the extrusion device heater.

Suppressing dust at 230 may include suppressing dust formed from and/or by the untreated mixture and may be performed during the grinding at 220. The suppressing at 230 may include suppressing the dust in any suitable manner. As an example, the suppressing at 230 may include adding water to the untreated mixture prior to and/or during the grinding at 220. As a result, and in some examples, the treated mixture may include water. In such examples, the treated mixture may include any suitable amount, proportion, and/or percentage of water, examples of which include at least 1 wt %, at least 2 wt %, at least 4 wt %, at least 6 wt %, at least 8 wt %, at least 10 wt %, at most 15 wt %, at most 20 wt % water, at most 30 wt %, at most 25 wt %, at most 20 wt %, at most 15 wt %, and/or at most 10 wt % water. In a specific example, the treated mixture may include approximately 5% water.

Measuring a particle size at 240 may include measuring, establishing, and/or determining the particle size, or an average particles size, of inorganic, sunscreen-active material within the treated mixture and may be performed subsequent to the grinding at 220. When methods 200 include the measuring at 240, methods 200 further may include repeating at least the heating at 210, the grinding at 220, and the measuring at 240 until the average particle size of inorganic, sunscreen-active material within the treated mixture is within a threshold particle size range. State differently, the repeating may be responsive to and/or a result of the average particle size of inorganic, sunscreen-active material within the treated mixture being greater than the threshold particle size range. Examples of the average particle size are disclosed herein.

Examples of the threshold particle size range include at least 50 nm, at least 100 nm, at least 200 nm, at least 300 nm, at least 400 nm, at least 500 nm, at least 600 nm, at least 700 nm, at least 800 nm, at least 900 nm, at least 1 micrometer, at least 2 micrometers, at least 4 micrometers, at least 6 micrometers, at least 8 micrometers, at least 10 micrometers, at most 20 micrometers, at most 18 micrometers, at most 16 micrometers, at most 15 micrometers, at most 14 micrometers, at most 12 micrometers, at most 10 micrometers, at most 8 micrometers, and/or at most 6 micrometers. In a specific example, the threshold particle size range may be between 100 nm and 15 micrometers.

Chipping the treated mixture at 250 may include chipping the treated mixture to form and/or to define a chipped treated mixture. This may include chipping the treated mixture to transform extrusions and/or sheets of the treated mixture, such as may be generated during the grinding at 220, into chips, examples of which are illustrated in FIG. 2 and indicated at 18. This may permit and/or facilitate storage, or long-term storage, of the treated mixture prior to incorporation of the treated mixture into a sunscreen composition, such as via performing methods 300.

Dewatering the treated mixture at 260 may include dewatering the treated mixture to form and/or to define a dewatered treated mixture. The dewatering at 260 may be performed in any suitable manner. As an example, the dewatering at 260 may include heating and drying the treated mixture and/or placing the treated mixture in a low-humidity environment. As another example, the dewatering at 260 may be a result of and/or may be performed during the heating at 210 and/or the grinding at 220.

The dewatering at 260 may be performed subsequent to the grinding at 220 and/or subsequent to the chipping at 250, such as to remove water utilized to suppress dust from the untreated mixture. When methods 200 include both the chipping at 250 and the dewatering at 260, the treated mixture may be referred to herein as a dewatered and chipped treated mixture.

Subsequent to formation, the treated mixture may be stored and/or placed in long-term storage prior to being incorporated into sunscreen compositions 10, as disclosed herein. If the treated mixture is stored for long periods of time, greater than 1 wt % water in the treated mixture may result in decomposition and/or bacterial contamination. Thus, the treated mixture may be measured for water; and, if the treated mixture contains greater than 1% water, the treated mixture may be heated and/or dried to achieve less than 1% water.

FIG. 5 is a flowchart illustrating examples of methods 300 of forming a sunscreen composition, according to the present disclosure. Examples of the sunscreen composition are disclosed herein with reference to sunscreen composition 10. Methods 300 include providing sunscreen-active material particles at 310 and dispersing the sunscreen-active material particles at 320.

Providing the sunscreen-active material particles at 310 may include providing any suitable sunscreen-active material particles, which may include inorganic sunscreen-active material 30, first water-soluble polymer 40, second water-soluble polymer 50, and surfactant 60. The providing at 310 may be performed in any suitable manner. As an example, the providing at 310 may include providing sunscreen-active mixture 22, which is disclosed herein. As another example, the providing at 310 may include providing the treated mixture, the dewatered treated mixture, the chipped treated mixture, and/or the dewatered and chipped treated mixture formed during methods 200. This may include performing any suitable step and/or steps of methods 200.

Dispersing the sunscreen-active material particles at 320 may include dispersing the sunscreen-active material particles within a cosmetic emulsion. Examples of the cosmetic emulsion are disclosed herein with reference to cosmetic emulsion 90. The dispersing at 320 may be performed in any suitable manner. As an example, the dispersing at 320 may include heating the cosmetic emulsion to a dispersing temperature that is greater than the first glass transition temperature and/or agitating the cosmetic emulsion. As another example, the dispersing at 320 may include adding the sunscreen-active material particles to the cosmetic emulsion and/or mixing the sunscreen-active material particles into the cosmetic emulsion, such as to define the sunscreen composition. As yet another example, and when the dispersing at 320 includes heating the cosmetic emulsion, the dispersing at 320 further may include cooling the sunscreen composition and/or permitting the sunscreen composition to cool.

The dispersing temperature may include and/or be any suitable temperature that is greater than the first glass transition temperature. Examples of a difference between the dispersing temperature and the first glass transition temperature include at least 2° C., at least 4° C., at least 6° C., at least 8° C., at least 10° C., at least 12° C., at least 14° C., at least 16° C., at least 18° C., at least 20° C., at least 25° C., at most 30° C., at most 28° C., at most 26° C., at most 24° C., at most 22° C., at most 20° C., at most 18° C., at most 16° C., at most 14° C., at most 12° C., and/or at most 10° C.

The following sections describe examples of sunscreen compositions 10 as well as related methods, according to the present disclosure. The examples in these sections are intended for illustration and should not be interpreted as limiting the scope of the present disclosure.

### EXAMPLE I

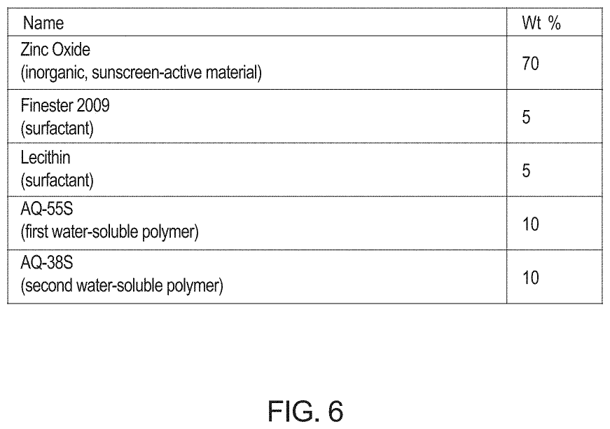

In a first example, and as indicated by the table illustrated in FIG. 6, a mixture containing an inorganic, sunscreen-active material 30, in the form of Zinc Oxide, two surfactants 60, in the form of Finester 2009 and Lecithin, a first water-soluble polymer 40, in the form of AQ-55S (i.e. polyester-5 with a glass transition temperature of 51-55° C.), and a second water-soluble polymer 50, in the form of AQ-38S (i.e., polyester-5 with a glass transition temperature of 35-38° C.), was prepared utilizing the illustrated relative composition. The mixture was blended in a sigma type blender at room temperature. Water, in the amount of up to 5% by weight of the blended mixture, was added to form a wet mixture (e.g., for dust suppression). The wet mixture was mixed in the homogenizer until homogenous.

The wet mixture then was removed from the homogenizer and rolled onto a two-roller Ferrell-Ross roller mill, with the front roller unheated and the back roller heated to 55° C., which is the glass transition temperature of AQ-55S. When passing through the heated roller, the wet mixture became like dry sheets. After passing through both rollers, the average particle size of the inorganic, sunscreen-active material within the dry sheets was measured with a hegman gauge. If the average particle size was greater than 8 microns, the wet mixture was passed through the roller mill again. The average particle size was measured again and the process was repeated until the average particle size was below 8 microns but above 1 micron. Water evaporated during this process. The dry sheets then were passed through a chipper to break the dry sheets up into chips. and the chips were stored (e.g., as sunscreen-active material particles 20).

Subsequently, the dry mixture was wet dispersed into an oil-in-water emulsion to create a sunscreen lotion, with the mass ratio of emulsion to dry mixture being approximately 6.1:1. The resulting sunscreen lotion exhibited approximately a 53 SPF with ZnO defining approximately 10.5 wt % of the final composition. In contrast, and as discussed, commercially available mineral sunscreens generally require a ZnO content of approximately 25 wt % to achieve a comparable SPF.

### EXAMPLE II

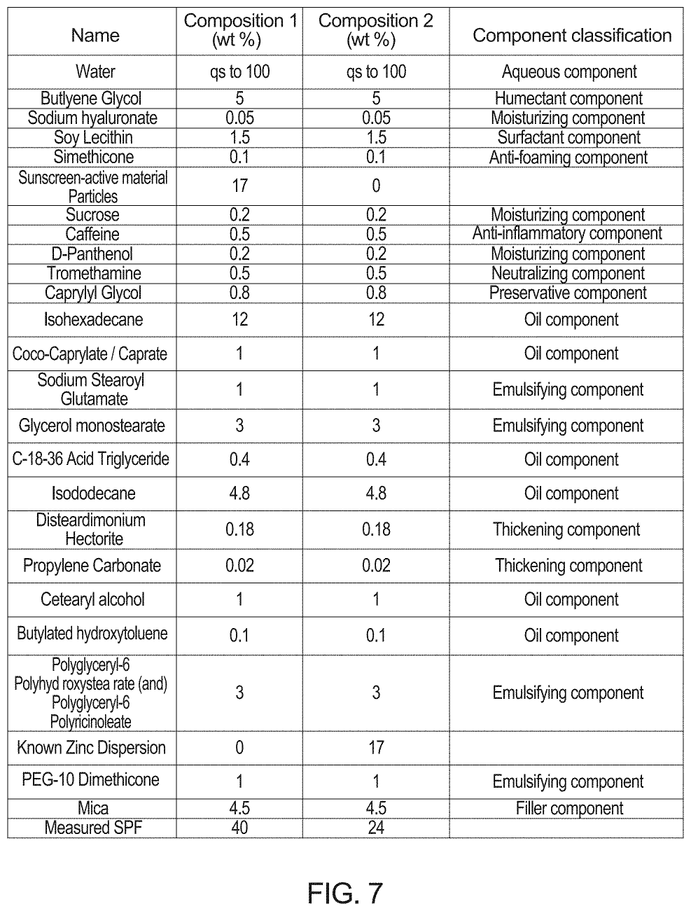

In a second example, and as indicated by the table illustrated in FIG. 7, two sunscreen compositions were prepared utilizing the illustrated relative compositions. The two compositions were identical with the exception that Composition 1 included 17 wt % sunscreen-active material particles 20, according to the present disclosure, and Composition 2 included 17 wt % of a known zinc dispersion commonly utilized in commercially available mineral sunscreens. Stated differently, Composition 1 was a sunscreen composition according to the present disclosure, while Composition 2 was a comparative sunscreen composition similar to commercially available mineral sunscreens that do not include and/or utilize sunscreen-active material particles according to the present disclosure. The two compositions were submitted to an analytical laboratory (Florida Suncare Testing, Inc.) for SPF testing. The results of this testing, as indicated on the last row in the table of FIG. 7, show that Composition 1 achieved an SPF of 40, while Composition 2 was only achieved an SPF of 24. Stated differently, the sunscreen composition according to the present disclosure (i.e., Composition 1, which includes sunscreen-active material particles 20 according to the present disclosure) provided a nearly 2× increase in SPF when compared to a similar sunscreen composition (i.e., Composition 2) that instead utilizes zinc oxide particles commonly utilized in commercially available mineral sunscreens.

### Discussion

Example I and Example II both illustrate that sunscreen compositions 10 that include sunscreen-active material particles 20, according to the present disclosure, provide significantly improved sun-blocking performance (i.e., higher SPF) when compared to commercially available mineral sunscreens. This improved sun-blocking performance is only one of several benefits of sunscreen compositions 10, and other such benefits are discussed herein.

Stated differently, sunscreen composition 10 and methods 200/300 for manufacture described herein provide several advantages over known solutions for creating a stable, efficient, safe, non-irritating, UV-blocking sunscreen composition. For example, illustrative embodiments and examples described herein allow for a lower weight percentage of inorganic, sunscreen-active material to achieve a comparable SPF than current sunscreens. For example, the sunscreen composition in Example I can achieve a SPF of 53 with only 10.5% ZnO by weight percentage.

Additionally, and among other benefits, illustrative embodiments and examples described herein allow a more efficient processing to produce desired particle sizes of sunscreen-active materials. Additionally, and among other benefits, illustrative embodiments and examples described herein allow for a more stable mixture, with generally less than 1% of water, which can be stored for relatively long periods of time with little bacterial contamination. This mixture can readily be put into a form usable on the skin by suspension, dispersion, or emulsion. The usable emulsion will have relatively little agglomeration of inorganic, sunscreen-active material particles. Moreover, the texture will be less sticky or thick than current mineral sunscreens.

Additionally, and among other benefits, illustrative embodiments and examples described herein allow for a sunscreen composition with less white cast when applied to the skin. No known system or device can perform these functions.

The effectiveness of sunscreen-active material particles 20 in blocking UV radiation is unexpected, because adding a polymer to the inorganic, sunscreen-active material generally would be expected to reduce electron movement within the inorganic, sunscreen-active material and, therefore, reduce the electron's ability to effectively absorb UV radiation. However, the above results clearly indicate that inorganic, sunscreen-active material 30 within sunscreen-active material particles 20 not only continues to block UV radiation but does so more effectively when compared to individual particles of inorganic, sunscreen-active material. Overall, the composition is surprisingly effective at blocking UV radiation, and can achieve a similar SPF with approximately half of the inorganic sunscreen-active material utilized in commercially available mineral sunscreens.

In the above discussions, broad and/or comprehensive ranges have been disclosed, both for the various components of sunscreen-active material particles 20 and for the various components of sunscreen compositions 10 that include sunscreen-active material particles 20. These broad ranges are disclosed because the invented sunscreen-active material particles may be utilized in a wide array of different sunscreen compositions and/or in other topically applied products for which UV-protection characteristics are desired. In addition, a desired level of UV protection may vary from product-to-product.

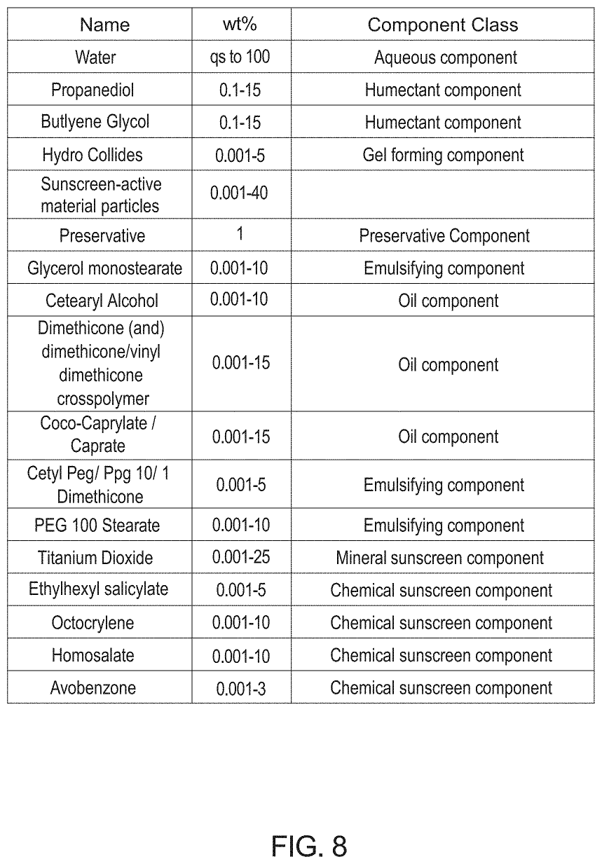

That said, FIGS. 8-12 provide tables that illustrate more specific examples of sunscreen compositions, according to the present disclosure. In FIGS. 7-11, the first column includes the INCI name for the variously included components, the second column describes nominal relative compositional amounts of the variously included components (as a weight percentage of the overall composition), and the third column describes the component class for the variously included components. In these more specific examples, another component from a given component class may be substituted for the indicated component without departing from the scope of the present disclosure. In addition, one more of the indicated components may be omitted from the sunscreen composition without departing from the present disclosure and/or one or more additional components, as disclosed herein, may be included in the sunscreen composition without departing from the scope of the present disclosure.

FIG. 8 is a table that describes components and relative compositional amounts for a hybrid sunscreen composition, according to the present disclosure. The hybrid sunscreen composition includes both the invented sunscreen-active material particles and one or more chemical sunscreen components.

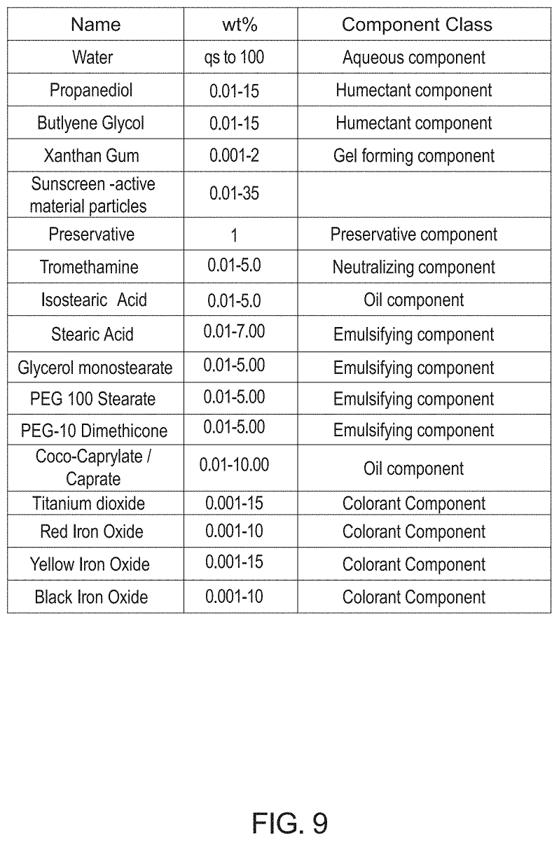

FIG. 9 is a table that describes components and relative compositional amounts for a color sunscreen composition, according to the present disclosure. For the compositions disclosed in FIG. 9, various amounts of the listed colorant components may be utilized to achieve a desired color and/or hue for the sunscreen composition, either before or after application to a user's skin.

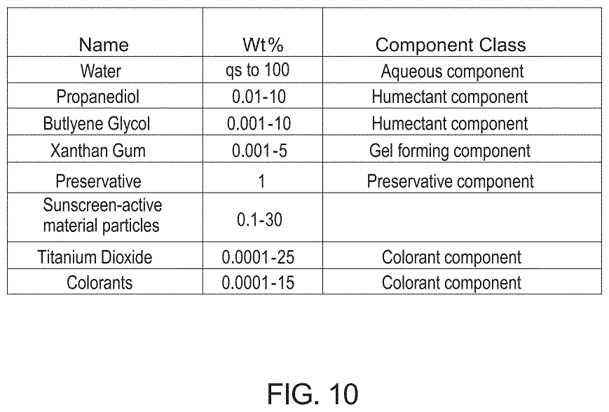

FIG. 10 is a table that describes components and relative compositional amounts for a spray sunscreen composition, according to the present disclosure. The compositions disclosed in FIG. 10 may be less viscous when compared to other compositions, which are disclosed herein, such as to permit and/or facilitate being sprayed onto the user's skin utilizing a pump and/or aerosol sprayer.

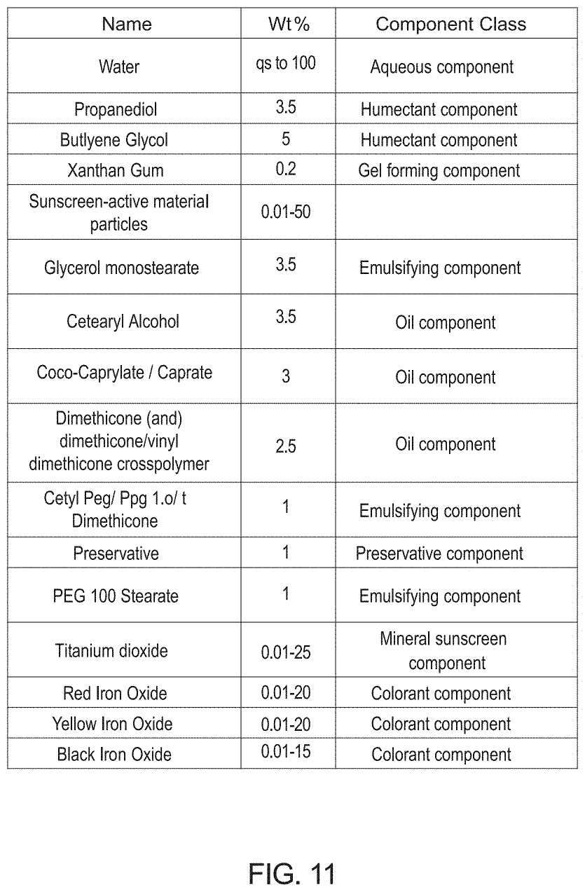

FIG. 11 is a table that describes components and relative compositional amounts for a cream sunscreen composition, according to the present disclosure. The compositions disclosed in FIG. 11 may exhibit a more creamy, or lotion-like texture when compared to one or more other sunscreen compositions according to the present disclosure.

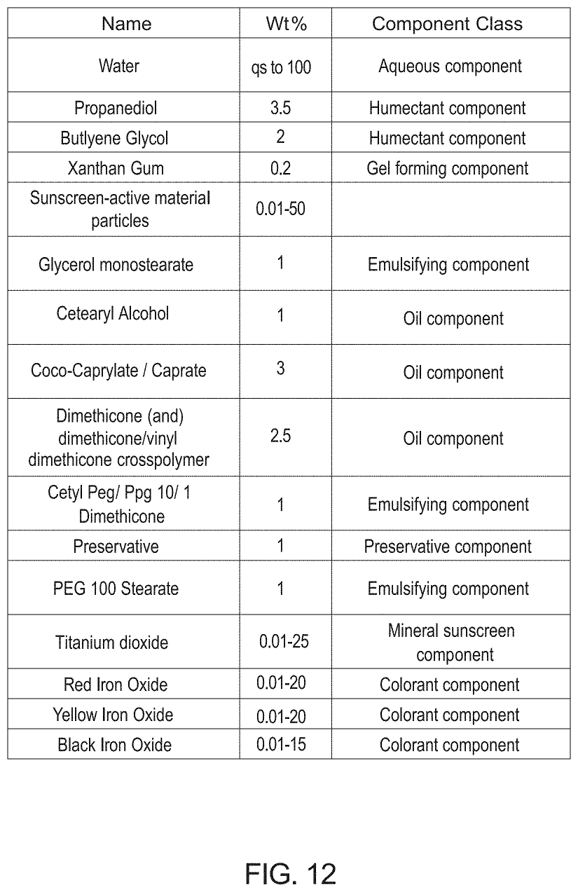

FIG. 12 is a table that describes components and relative compositional amounts for a serum sunscreen composition, according to the present disclosure. The compositions disclosed in FIG. 12 may be lighter, may be less viscous, and/or may be more readily absorbed into the user's skin when compared to one or more other sunscreen compositions according to the present disclosure.

In the present disclosure, several of the illustrative, non-exclusive examples have been discussed and/or presented in the context of flow diagrams, or flow charts, in which the methods are shown and described as a series of blocks, or steps. Unless specifically set forth in the accompanying description, it is within the scope of the present disclosure that the order of the blocks may vary from the illustrated order in the flow diagram, including with two or more of the blocks (or steps) occurring in a different order and/or concurrently.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” may refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

As used herein, the phrase “at least one,” in reference to a list of one or more entities should be understood to mean at least one entity selected from any one or more of the entities in the list of entities, but not necessarily including at least one of each and every entity specifically listed within the list of entities and not excluding any combinations of entities in the list of entities. This definition also allows that entities may optionally be present other than the entities specifically identified within the list of entities to which the phrase “at least one” refers, whether related or unrelated to those entities specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including entities other than B); in another embodiment, to at least one, optionally including more than one, B, with no A present (and optionally including entities other than A); in yet another embodiment, to at least one, optionally including more than one, A, and at least one, optionally including more than one, B (and optionally including other entities). In other words, the phrases “at least one,” “one or more,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C,” and “A, B, and/or C” may mean A alone, B alone, C alone, A and B together, A and C together, B and C together, A, B, and C together, and optionally any of the above in combination with at least one other entity.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

As used herein the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function but that the element, component, and/or other subject matter is specifically selected, created, implemented, utilized, programmed, and/or designed for the purpose of performing the function. It is also within the scope of the present disclosure that elements, components, and/or other recited subject matter that is recited as being adapted to perform a particular function may additionally or alternatively be described as being configured to perform that function, and vice versa.

As used herein, the phrase, “for example,” the phrase, “as an example,” and/or simply the term “example,” when used with reference to one or more components, features, details, structures, embodiments, and/or methods according to the present disclosure, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

As used herein, “at least substantially,” when modifying a degree or relationship, may include not only the recited “substantial” degree or relationship, but also the full extent of the recited degree or relationship. A substantial amount of a recited degree or relationship may include at least 75% of the recited degree or relationship. For example, an object that is at least substantially formed from a material includes objects for which at least 75% of the objects are formed from the material and also includes objects that are completely formed from the material. As another example, a first length that is at least substantially as long as a second length includes first lengths that are within 75% of the second length and also includes first lengths that are as long as the second length.

As used herein, the terms “comprising,” “including,” and “having” (and conjugations thereof) are used interchangeably to mean including but not necessarily limited to, and are open-ended terms not intended to exclude additional, unrecited elements or method steps.

As used herein, the terms such as “first”, “second”, “third”, etc. are used to distinguish or identify various members of a group, or the like, and are not intended to show serial or numerical limitation.

As used herein, the term “providing,” in the context of a method, may include receiving, obtaining, purchasing, manufacturing, generating, processing, preprocessing, and/or the like, such that the object or material provided is in a state and configuration for other steps to be carried out.

As used herein, the term “approximately”, when used in terms of a weight percentages, means “within ten percent above and below the given value.” For example, “approximately 20%” means “18-22%” while “approximately 50%” means “45-55%.” “Approximately”, when used in terms of temperature, means “within 2° C. above and below a given value.” “Approximately”, when used in terms of ratios, means “within 10% of the quotient.”

Illustrative, non-exclusive examples of sunscreen-active mixtures, sunscreen compositions, and methods according to the present disclosure are presented in the following enumerated paragraphs. It is within the scope of the present disclosure that an individual step of a method recited herein, including in the following enumerated paragraphs, may additionally or alternatively be referred to as a “step for” performing the recited action.

A0. A sunscreen-active mixture, the mixture comprising:

an inorganic, sunscreen-active material;

a first water-soluble polymer that has a first glass transition temperature, optionally of at least 45° C.;

a second water-soluble polymer that has a second glass transition temperature that is less than the first glass transition temperature; and

a surfactant.

A1. The mixture of paragraph A0, wherein the inorganic, sunscreen-active material includes, consists of, or consists essentially of at least one, and optionally both, of zinc oxide (ZnO) and titanium dioxide (TiO₂).

A2. The mixture of any of paragraphs A0-A1, wherein the mixture includes at least one of:

at least 1 weight percent (wt %), at least 2 wt %, at least 4 wt %, at least 6 wt %, at least 8 wt %, at least 10 wt %, at least 15 wt %, at least 20 wt %, at least 25 wt %, at least 30 wt %, at least 40 wt %, at least 45 wt %, at least 50 wt %, at least 55 wt %, at least 60 wt %, at least 65 wt %, at least 70 wt %, at least 75 wt %, or at least 80 wt % of the inorganic, sunscreen-active material; and

at most 90 wt %, at most 85 wt %, at most 80 wt %, at most 75 wt %, at most 70 wt %, at most 60 wt %, at most 50 wt %, at most 40 wt %, at most 30 wt %, at most 20 wt %, or at most 10 wt % of the inorganic, sunscreen-active material.

A3. The mixture of any of paragraphs A0 through A2, wherein the inorganic, sunscreen-active material comprises approximately 70 wt % of the mixture.

A4. The mixture of any of paragraphs A0 through A3, wherein at least one, and optionally both, of the first water-soluble polymer and the second water-soluble polymer includes, consists of, or consists essentially of at least one of a polyester and a polyesteramide.

A5. The mixture of any of paragraphs A0 through A4, wherein at least one, and optionally both, of the first water-soluble polymer and the second water-soluble polymer includes, consists of, or consists essentially of an aromatic sulfopolyester.

A6. The mixture of any of paragraphs A0-A5, wherein at least one, and optionally both, of the first water-soluble polymer and the second water-soluble polymer includes, consists of, or consists essentially of a polymeric resin.

A7. The mixture of any of paragraphs A0-A6, wherein a difference between the first glass transition temperature and the second glass transition temperature is at least one of:

at least 2° C., at least 4° C., at least 6° C., at least 8° C., at least 10° C., at least 12° C., at least 14° C., at least 16° C., at least 18° C., or at least 20° C.; and

at most 30° C., at most 28° C., at most 26° C., at most 24° C., at most 22° C., at most 20° C., at most 18° C., at most 16° C., at most 14° C., at most 12° C., or at most 10° C.

A8. The mixture of any of paragraphs A0-A7, wherein the first glass transition temperature is at least one of:

at least 35° C., at least 40° C., at least 45° C., at least 50° C., at least 55° C., at least 60° C., at least 65° C., at least 70° C., at least 75° C., or at least 80° C.; and

at most 90° C., at most 85° C., at most 80° C., at most 75° C., at most 70° C., at most 65° C., at most 60° C., at most 55° C., at most 50° C., at most 45° C., or at most 40° C.

A9. The mixture of any of paragraphs A0-A8, wherein the first glass transition temperature is approximately 55° C.

A10. The mixture of any of paragraphs A0-A9, wherein the second glass transition temperature is at least one of:

at least 25° C., at least 30° C., at least 35° C., at least 40° C., at least 45° C., at least 50° C., at least 55° C., at least 60° C., at least 65° C., or at least 70° C.; and

at most 80° C., at most 75° C., at most 70° C., at most 65° C., at most 60° C., at most 55° C., at most 50° C., at most 45° C., at most 40° C., at most 35° C., or at most 30° C.

A11. The mixture of any of paragraphs A0-A10, wherein the second glass transition temperature is approximately 38° C.

A12. The mixture of any of paragraphs A0-A11, wherein the first water-soluble polymer includes, consists of, or consists essentially of at least one of AQ™ 55S, a polyester-5 with a glass transition temperature of, or of approximately, 51-55° C., a sulfopolyester with a glass transition temperature of, or of approximately, 51-55° C., and a copolymer of diethylene glycol, 1,4-cyclohexanedimethanol and simple esters of isophthalic acid and sulfoisophthalic acid with a glass transition temperature of, or of approximately, 51-55° C.

A13. The mixture of any of paragraphs A0-A12, wherein the second water-soluble polymer includes, consists of, or consists essentially of at least one of AQ™ 38S, a polyester-5 with a glass transition temperature of, or of approximately, 35-38° C., a sulfopolyester with a glass transition temperature of, or of approximately, 35-38° C., and a copolymer of diethylene glycol, 1,4-cyclohexanedimethanol and simple esters of isophthalic acid and sulfoisophthalic acid with a glass transition temperature of, or of approximately, 35-38° C.

A14. The mixture of any of paragraphs A0-A13, wherein the mixture includes at least one of:

at least 0.001 wt %, at least 0.01 wt %, at least 0.1 wt %, at least 1 wt %, at least 2 wt %, at least 4 wt %, at least 6 wt %, at least 8 wt %, at least 10 wt %, at least 15 wt %, at least 20 wt %, at least 25 wt %, at least 30 wt %, at least 35 wt %, at least 40 wt %, at least 45 wt %, or at least 50 wt % of the first water-soluble polymer; and

at most 60 wt %, at most 55 wt %, at most 50 wt %, at most 45 wt %, at most 40 wt %, at most 35 wt %, at most 30 wt %, at most 25 wt %, at most 20 wt %, at most 18 wt %, at most 16 wt %, at most 14 wt %, at most 12 wt %, or at most 10 wt % of the first water-soluble polymer.

A15. The mixture of any of paragraphs A0-A14, wherein the first water-soluble polymer comprises approximately 10 wt % of the mixture.

A16. The mixture of any of paragraphs A0-A15, wherein the mixture includes at least one of:

at least 0.001 wt %, at least 0.01 wt %, at least 0.1 wt %, at least 1 wt %, at least 2 wt %, at least 4 wt %, at least 6 wt %, at least 8 wt %, at least 10 wt %, at least 15 wt %, at least 20 wt %, at least 25 wt %, at least 30 wt %, at least 35 wt %, or at least 40 wt % of the second water-soluble polymer; and

at most 50 wt %, at most 45 wt %, at most 40 wt %, at most 35 wt %, at most 30 wt %, at most 25 wt %, at most 20 wt %, at most 18 wt %, at most 16 wt %, at most 14 wt %, at most 12 wt %, or at most 10 wt % of the second water-soluble polymer.

A17. The mixture of any of paragraphs A0-A16, wherein the second water-soluble polymer comprises approximately 10 wt % of the mixture.

A18. The mixture of any of paragraphs A0-A17, wherein the surfactant includes, consists of, or consists essentially of at least one, at least two, at least three, at least four, or at least five of an anionic surfactant, a cationic surfactant, an amphoteric surfactant, an ethoxylated surfactant, a lecithin, soy lecithin, Finester 2009, polyglyceryl-3 lactate/laurate, polyglyceryl-3 laurate, polyglycerin-3, and polyglycerin-3 (and) polyglyceryl-3 Lactate/Laurate

A19. The mixture of any of paragraphs A0-A18, wherein the mixture includes at least one of:

at least 0.001 wt %, at least 0.01 wt %, at least 0.1 wt %, at least 1 wt %, at least 2 wt %, at least 4 wt %, at least 6 wt %, at least 8 wt %, at least 10 wt %, at least 15 wt %, at least 20 wt %, at least 25 wt %, or at least 30 wt % of the surfactant; and

at most 40 wt %, at most 35 wt %, at most 30 wt %, at most 25 wt %, at most 20 wt %, at most 18 wt %, at most 16 wt %, at most 14 wt %, at most 12 wt %, or at most 10 wt % of the surfactant.

A20. The mixture of any of paragraphs A0-A19, wherein the surfactant comprises approximately 10 wt % of the mixture.

A21. The mixture of any of paragraphs A0-A20, wherein the surfactant includes a first surfactant and a second surfactant.

A22. The mixture of any of paragraph A21, wherein the first surfactant includes, consists of, or consists essentially of at least one of a lecithin and soy lecithin.

A23. The mixture of any of paragraphs A20-A22, wherein the second surfactant includes, consists of, or consists essentially of at least one, at least two, or at least three of Finester 2009, polyglyceryl-3 lactate/laurate, polyglyceryl-3 laurate, polyglycerin-3, and polyglycerin-3 (and) polyglyceryl-3 Lactate/Laurate.

A24. The mixture of any of paragraphs A21-A23, wherein a mass ratio of the first surfactant to the second surfactant, within the mixture, is 2:3 to 3:2.

A25. The mixture of any of paragraphs A21-A24, wherein a/the mass ratio of the first surfactant to the second surfactant, within the mixture, is approximately 1:1.

A26. The mixture of any of paragraphs A0 through A25, further comprising iron oxide.

A27. The mixture of paragraph A26, wherein the mixture includes at least one of:

at least 0.01 wt %, at least 0.1 wt %, at least 1 wt %, at least 2 wt %, at least 4 wt %, at least 6 wt %, at least 8 wt %, at least 10 wt %, at least 15 wt %, at least 20 wt %, at least 25 wt %, at least 30 wt %, at least 35 wt %, or at least 40 wt % iron oxide; and

at most 50 wt %, at most 45 wt %, at most 40 wt %, at most 35 wt %, at most 30 wt %, at most 25 wt %, at most 20 wt %, at most 18 wt %, at most 16 wt %, at most 14 wt %, at most 12 wt %, at most 10 wt %, at most 8 wt %, at most 6 wt %, at most 4 wt %, or at most 2 wt % iron oxide.

A28. The mixture of any of paragraphs A0-A27, wherein the mixture further includes water.

A29. The mixture of any of paragraphs A0-A28, wherein the mixture includes at least one of:

(i) at least 1 wt %, at least 2 wt %, at least 4 wt %, at least 6 wt %, at least 8 wt %, at least 10 wt %, at most 15 wt %, or at most 20 wt % water; and

(ii) at most 30 wt %, at most 25 wt %, at most 20 wt %, at most 15 wt %, or at most 10 wt % water.

A30. The mixture of any of paragraphs A0-A29, wherein the mixture includes approximately 5 wt % water.

A31. The mixture of any of paragraphs A0-A30, wherein the inorganic, sunscreen-active material is a particulate inorganic, sunscreen-active material that include a plurality of material particles.

A32. The mixture of paragraph A31, wherein an average particle size of the plurality of material particles is at least one of:

(i) at least 50 nanometers (nm), at least 100 nm, at least 200 nm, at least 300 nm, at least 400 nm, at least 500 nm, at least 600 nm, at least 700 nm, at least 800 nm, at least 900 nm, at least 1 micrometer, at least 2 micrometers, at least 4 micrometers, at least 6 micrometers, at least 8 micrometers, or at least 10 micrometers; and

(ii) at most 20 micrometers, at most 18 micrometers, at most 16 micrometers, at most 15 micrometers, at most 14 micrometers, at most 12 micrometers, at most 10 micrometers, at most 8 micrometers, or at most 6 micrometers.

A33. The mixture of any of paragraphs A31-A32, wherein the plurality of material particles are embedded within a mixture of the first water-soluble polymer, the second water-soluble polymer, and the surfactant.

B0. A method of manufacturing a treated mixture for a sunscreen composition, the method comprising:

heating an untreated mixture, which includes, consists of, consists essentially of, or is the mixture of any of paragraphs A0 through A333, to a heated temperature that is within a threshold temperature differential of the first glass transition temperature of the first water-soluble polymer, optionally wherein the heated temperature is greater than the second glass transition temperature of the second water-soluble polymer; and

grinding the untreated mixture to generate the treated mixture.

B1. The method of paragraph B0, wherein the heating is at least one of:

(i) at least partially concurrent with the grinding; and

(ii) completely concurrent with the grinding.

B2. The method of any of paragraphs B0-B1, wherein the heating includes maintaining the untreated mixture at the heated temperature during the grinding.

B3. The method of any of paragraphs B0-B2, wherein the threshold temperature differential is at most 0.5° C., at most 1° C., at most 1.5° C., at most 2° C., at most 3° C., at most 4° C., at most 5° C., at most 6° C., at most 7° C., at most 8° C., at most 9° C., or at most 10° C.

B4. The method of any of paragraphs B0-B3, wherein the heating includes maintaining the untreated mixture below the first glass transition temperature during the grinding.

B5. The method of any of paragraphs B0-B4, wherein the grinding includes grinding on a roller mill with at least two rollers.

B6. The method of paragraph B5, wherein a first roller of the at least two rollers is unheated, and further wherein a second roller of the at least two rollers is heated to a roller temperature that is within the threshold temperature differential of the first glass transition temperature of the first water-soluble polymer.

B7. The method of any of paragraphs B0-B4, wherein the grinding includes extruding the untreated mixture within an extrusion device.

B8. The method of paragraph B7, wherein the extrusion device includes an extrusion device heater, and further wherein the heating includes heating the untreated mixture utilizing the extrusion device heater.

B9. The method of any of paragraphs B0-B8, wherein the method further includes measuring an average particle size of the treated mixture subsequent to the grinding.

B10. The method of paragraph B9, further comprising repeating the heating, the grinding, and the measuring the average particle size of the treated mixture until the average particle size of the treated mixture is within a threshold particle size range.

B11. The method of paragraph B10, wherein the repeating is responsive to the average particle size of the treated mixture, as determined during the measuring the average particle size of the treated mixture, being greater than the threshold particle size range.

B12. The method of any of paragraphs B10-B11, wherein the threshold particle size range is at least one of:

(i) at least 50 nm, at least 100 nm, at least 200 nm, at least 300 nm, at least 400 nm, at least 500 nm, at least 600 nm, at least 700 nm, at least 800 nm, at least 900 nm, at least 1 micrometer, at least 2 micrometers, at least 4 micrometers, at least 6 micrometers, at least 8 micrometers, or at least 10 micrometers; and

(ii) at most 20 micrometers, at most 18 micrometers, at most 16 micrometers, at most 15 micrometers, at most 14 micrometers, at most 12 micrometers, at most 10 micrometers, at most 8 micrometers, or at most 6 micrometers.

B13. The method of any of paragraphs B0-B12, wherein, during the grinding, the method further includes suppressing dust from the untreated mixture.

B14. The method of paragraph B13, wherein the suppressing includes adding water to the untreated mixture.

B15. The method of any of paragraphs B0-B14, wherein the treated mixture further includes water.

B16. The method of any of paragraphs B0-B15, wherein the treated mixture includes at least one of:

(i) at least 1 wt %, at least 2 wt %, at least 4 wt %, at least 6 wt %, at least 8 wt %, at least 10 wt %, at most 15 wt %, or at most 20 wt % water; and

(ii) at most 30 wt %, at most 25 wt %, at most 20 wt %, at most 15 wt %, or at most 10 wt % water.

B17. The method of any of paragraphs B1-B8, wherein the treated mixture includes approximately 5% water.

B18. The method of any of paragraphs B14-B17, subsequent to the grinding, the method further includes dewatering the treated mixture to define a dewatered treated mixture.

B19. The method of claim 18, wherein the dewatering includes heating and drying the treated mixture.

B20. The method of any of paragraphs B0-B19, wherein the method further includes chipping the treated mixture to define a chipped treated mixture.

C0. A sunscreen composition, comprising:

sunscreen-active material particles that include, consist of, or consist essentially of at least one of:

(i) the mixture of any of paragraphs A0-A32;

(ii) the treated mixture formed via the method of any of paragraphs B0-B20;

(iii) the dewatered treated mixture formed via the method of any of paragraphs B18-B19;

(iv) the chipped treated mixture formed via the method of paragraph B20; and

(v) a dewatered and chipped treated mixture formed via the method of paragraph B20 when dependent from paragraph B19; and

a cosmetic emulsion.

C1. The sunscreen composition of paragraph C0, wherein the sunscreen-active material particles are dispersed, or suspended, within the cosmetic emulsion.

C2. The sunscreen composition of any of paragraphs C0-C1, wherein the sunscreen-active material particles include individual sunscreen-active material particles dispersed, or suspended, within the cosmetic emulsion.

C3. The sunscreen composition of any of paragraphs C0-C2, wherein sunscreen composition includes at least one of:

(i) at least 0.001 wt %, at least 0.01 wt %, at least 0.1 wt %, at least 1 wt %, at least 2 wt %, at least 3 wt %, at least 4 wt %, at least 5 wt %, at least 6 wt %, at least 8 wt %, at least 10 wt %, at least 15 wt %, at least 20 wt %, at least 25 wt %, at least 30 wt %, at least 35 wt %, or at least 40 wt % of the sunscreen-active material particles; and

(ii) at most 50 wt %, at most 45 wt %, at most 40 wt %, at most 35 wt %, at most 30 wt %, at most 25 wt %, at most 20 wt %, at most 15 wt % or at most 10 wt % of the sunscreen-active material particles.

C4. The sunscreen composition of any of paragraphs C0-C3, wherein the sunscreen composition includes at least one of:

(i) at least 50 wt %, at least 55 wt %, at least 60 wt %, at least 65 wt %, at least 70 wt %, at least 80 wt %, at least 85 wt %, or at least 90 wt % of the cosmetic emulsion; and

(ii) at most 99.9 wt %, at most 99 wt %, at most 95 wt %, at most 90 wt %, at most 85 wt %, at most 80 wt %, at most 75 wt %, at most 70 wt %, at most 65 wt %, or at most 60 wt % of the cosmetic emulsion.

C5. The sunscreen composition of any of paragraphs C0-C6, wherein the sunscreen composition further includes an oil component.

C6. The sunscreen composition of paragraph C5, wherein at least one of the cosmetic emulsion includes the oil component, the oil component is dispersed within the cosmetic emulsion, and the oil component is suspended within the cosmetic emulsion.

C7. The sunscreen composition of any of paragraphs C5-C6, wherein the oil component includes, consists of, or consists essentially of at least one of a natural oil, a vegetable oil, coconut oil, a hydrocarbon, a hydrocarbon oil, a mineral oil, hydrogenated polyisobutene, a fatty alcohol, octyldodecanol, an ester, a C12-C15 alkyl benzoate, a C12 alkyl benzoate, a C13 alkyl benzoate, a C14 alkyl benzoate, a C15 alkyl benzoate, a diester, propylene glycol dipelargonate,a triester, glyceryl trioctanoate, a sterol derivative, lanolin, a wax, an animal waxes, beeswax, a plant wax, carnauba wax, a mineral wax, ozokerite, a petroleum wax, paraffin wax, a synthetic wax, polyethylene, a silicone, a volatile silicone, cyclomethicone, a polymeric silicone, dimethicone, an alkylated derivatives of polymeric silicones, cetyl dimethicone, lauryl trimethicone, a hydroxylated derivatives of polymeric silicones, dimethiconol, cetearyl alcohol, dimethicone/vinyl dimethicone crosspolymer, coco-caprylate/caprate, isohexadecane, C18-36 acid triglyceride, isododecane, butylated hydroxy-toluene, and isostearic acid.

C8. The sunscreen composition of any of paragraphs C5-C7, wherein the sunscreen composition includes at least one of:

(i) at least 0.001 wt %, at least 0.01 wt %, at least 0.1 wt %, at least 1 wt %, at least 2 wt %, at least 3 wt %, at least 4 wt %, at least 5 wt %, at least 6 wt %, at least 8 wt %, at least 10 wt %, at least 15 wt %, at least 20 wt %, at least 25 wt %, at least, or at least 30 wt % of the oil component; and

(ii) at most 40 wt %, at most 35 wt %, at most 30 wt %, at most 25 wt %, at most 20 wt %, at most 15 wt %, or at most 10 wt % of the oil component.

C9. The sunscreen composition of any of paragraphs C0-C8, wherein the sunscreen composition further includes an aqueous component.

C10. The sunscreen composition of paragraph C9, wherein at least one of the cosmetic emulsion includes the aqueous component, the aqueous component is dispersed within the cosmetic emulsion, and the aqueous component is suspended within the cosmetic emulsion.

C11. The sunscreen composition of any of paragraphs C9-C10, wherein the aqueous component includes, consists of, or consists essentially of water.

C12. The sunscreen composition of any of paragraphs C9-C11, wherein the sunscreen composition includes at least one of:

(i) at least 1 wt %, at least 2 wt %, at least 3 wt %, at least 4 wt %, at least 5 wt %, at least 6 wt %, at least 8 wt %, at least 10 wt %, at least 15 wt %, at least 20 wt %, at least 25 wt %, at least, at least 30 wt %, at least 35 wt %, at least 40 wt %, at least 45 wt %, or at least 50 wt % of the aqueous component; and

(ii) at most 80 wt %, at most 70 wt %, at most 60 wt %, at most 50 wt %, at most 40 wt %, at most 30 wt %, at most 20 wt %, at most 10 wt %, or at most 5 wt % of the aqueous component.

C13. The sunscreen composition of any of paragraphs C0-C12, wherein the sunscreen composition further includes an emulsifying component.

C14. The sunscreen composition of paragraph C9, wherein at least one of the cosmetic emulsion includes the emulsifying component, the emulsifying component is dispersed within the cosmetic emulsion, and the emulsifying component is suspended within the cosmetic emulsion.

C15. The sunscreen composition of any of paragraphs C13-C4, wherein the emulsifying component includes, consists of, or consists essentially of at least one of an oil-in-water emulsifier, an emulsifier having a hydrophilic lipophilic balance (HLB) of at least 6, a sorbitol derivative, sorbitan monolaurate, polysorbate 20, an ethoxylated alcohol, laureth-23, an ethoxylated fatty acid, PEG-1000 stearate, an amidoamine derivative, stearamidoethyl diethylamine, a sulfate ester, sodium lauryl sulfate, a phosphate ester, DEA cetyl phosphate, a fatty acid amine salt, TEA stearate, a water-in-oil emulsifier, an emulsifier having a hydrophilic lipophilic balance of 2 to 6, a sorbitan derivative, sorbitan laurate, sorbitan palmitate, an alkoxylated alcohol, laureth-4, a hydroxylated derivative of polymeric silicones, dimethicone copolyol, an alkylated derivatives of hydroxylated polymeric silicones, cetyl dimethicone copolyol, a glyceryl ester, polyglyceryl-4 isostearate, a beeswax derivative, sodium isostearoyl-2 lactylate, lecithin, glycerol monostearate, cetyl PEG/PPG-10/1 dimethicone, PEG 100 Stearate, sodium stearoyl glutamate, polyglyceryl-6 polyhydroxystearate, polyglyceryl-6 polyricinoleate, PEG-10 Dimethicone, and stearic acid.

C16. The sunscreen composition of any of paragraphs C13-C15, wherein the sunscreen composition includes at least one of:

(i) at least 0.001 wt %, at least 0.01 wt %, at least 0.1 wt %, at least 1 wt %, at least 2 wt %, at least 3 wt %, at least 4 wt %, at least 5 wt %, at least 6 wt %, at least 8 wt %, at least 10 wt %, at least 15 wt %, or at least 20 wt % of the emulsifying component; and

(ii) at most 30 wt %, at most 25 wt %, at most 20 wt %, at most 18 wt %, at most 16 wt %, at most 14 wt %, at most 12 wt %, at most 10 wt %, at most 8 wt %, at most 6 wt %, at most 4 wt %, at most 2 wt %, or at most 1 wt % of the emulsifying component.

C17. The sunscreen composition of any of paragraphs C0-C16, wherein the sunscreen composition further includes a humectant component.

C18. The sunscreen composition of paragraph C17, wherein at least one of the cosmetic emulsion includes the humectant component, the humectant component is dispersed within the cosmetic emulsion, and the humectant component is suspended within the cosmetic emulsion.

C19. The sunscreen composition of any of paragraphs C17-C18, wherein the humectant component includes, consists of, or consists essentially of at least one of propanediol and butylene glycol.

C20. The sunscreen composition of any of paragraphs C17-C19, wherein the sunscreen composition includes at least one of:

(i) at least 0.01 wt %, at least 0.1 wt %, at least 1 wt %, at least 2 wt %, at least 3 wt %, at least 4 wt %, at least 5 wt %, at least 6 wt %, at least 8 wt %, at least 10 wt %, at least 15 wt %, or at least 20 wt % of the humectant component; and

(ii) at most 30 wt %, at most 25 wt %, at most 20 wt %, at most 18 wt %, at most 16 wt %, at most 14 wt %, at most 12 wt %, at most 10 wt %, at most 8 wt %, at most 6 wt %, at most 4 wt %, at most 2 wt %, or at most 1 wt % of the humectant component.

C21. The sunscreen composition of any of paragraphs C0-C20, wherein the sunscreen composition further includes a gel-forming component.

C22. The sunscreen composition of paragraph C21, wherein at least one of the cosmetic emulsion includes the gel-forming component, the gel-forming component is dispersed within the cosmetic emulsion, and the gel-forming component is suspended within the cosmetic emulsion.

C23. The sunscreen composition of any of paragraphs C21-C22, wherein the gel-forming component includes, consists of, or consists essentially of at least one of a hydrocollide and xanthan gum.

C24. The sunscreen composition of any of paragraphs C21-C23, wherein the sunscreen composition includes at least one of:

(i) at least 0.001 wt %, at least 0.01 wt %, at least 0.1 wt %, at least 1 wt %, at least 2 wt %, at least 3 wt %, or at least 4 wt % of the gel-forming component; and

(ii) at most 6 wt %, at most 5 wt %, at most 4 wt %, at most 3 wt %, at most 2 wt %, or at most 1 wt % of the gel-forming component.

C25. The sunscreen composition of any of paragraphs C0-C24, wherein the sunscreen composition further includes a chemical sunscreen component.

C26. The sunscreen composition of paragraph C25, wherein at least one of the cosmetic emulsion includes the chemical sunscreen component, the chemical sunscreen component is dispersed within the cosmetic emulsion, and the chemical sunscreen component is suspended within the cosmetic emulsion.

C27. The sunscreen composition of any of paragraphs C25-C26, wherein the chemical sunscreen component includes, consists of, or consists essentially of at least one of ethylhexyl salicylate, octocrylene, homosalate, and avobenzone.

C28. The sunscreen composition of any of paragraphs C25-C27, wherein the sunscreen composition includes at least one of:

(i) at least 0.001 wt %, at least 0.01 wt %, at least 0.1 wt %, at least 1 wt %, at least 2 wt %, at least 3 wt %, at least 4 wt %, at least 5 wt %, at least 6 wt %, at least 8 wt %, at least 10 wt %, at least 15 wt %, or at least 20 wt % of the chemical sunscreen component; and

(ii) at most 30 wt %, at most 25 wt %, at most 20 wt %, at most 18 wt %, at most 16 wt %, at most 14 wt %, at most 12 wt %, at most 10 wt %, at most 8 wt %, at most 6 wt %, at most 4 wt %, at most 2 wt %, or at most 1 wt % of the chemical sunscreen component.

C29. The sunscreen composition of any of paragraphs C0-C28, wherein the sunscreen composition further includes a colorant component.

C30. The sunscreen composition of paragraph C29, wherein at least one of the cosmetic emulsion includes the colorant component, the colorant component is dispersed within the cosmetic emulsion, and the colorant component is suspended within the cosmetic emulsion.

C31. The sunscreen composition of any of paragraphs C29-C30, wherein the colorant component includes, consists of, or consists essentially of at least one of titanium dioxide, red iron oxide, yellow iron oxide, black iron oxide, an iron oxide, an FD&C dye, lake, and iron blue.

C32. The sunscreen composition of any of paragraphs C29-C31, wherein the sunscreen composition includes at least one of:

(i) at least 0.001 wt %, at least 0.01 wt %, at least 0.1 wt %, at least 1 wt %, at least 2 wt %, at least 3 wt %, at least 4 wt %, at least 5 wt %, at least 6 wt %, at least 8 wt %, at least 10 wt %, at least 15 wt %, at least 20 wt %, at least 30 wt %, or at least 40 wt % of the colorant component; and

(ii) at most 60 wt %, at most 55 wt %, at most 50 wt %, at most 45 wt %, at most 40 wt %, at most 35 wt %, at most 30 wt %, at most 25 wt %, at most 20 wt %, at most 18 wt %, at most 16 wt %, at most 14 wt %, at most 12 wt %, at most 10 wt %, at most 8 wt %, at most 6 wt %, at most 4 wt %, at most 2 wt %, or at most 1 wt % of the colorant component.

C33. The sunscreen composition of any of paragraphs C0-C32, wherein the sunscreen composition further includes a preservative component.

C34. The sunscreen composition of paragraph C33, wherein at least one of the cosmetic emulsion includes the preservative component, the preservative component is dispersed within the cosmetic emulsion, and the preservative component is suspended within the cosmetic emulsion.

C35. The sunscreen composition of any of paragraphs C33-C34, wherein the preservative component includes, consists of, or consists essentially of caprylyl glycol.

C36. The sunscreen composition of any of paragraphs C33-C35, wherein the sunscreen composition includes at least one of:

(i) at least 0.001 wt %, at least 0.01 wt %, at least 0.1 wt %, at least 0.2 wt %, at least 0.3 wt %, at least 0.4 wt %, at least 0.5 wt %, at least 0.6 wt %, at least 0.7 wt %, at least 0.8 wt %, at least 0.9 wt %, or at least 1 wt % of the preservative component; and

(ii) at most 2 wt %, at most 1.8 wt %, at most 1.6 wt %, at most 1.4 wt %, at most 1.2 wt %, at most 1 wt %, at most 0.9 wt %, at most 0.8 wt %, at most 0.7 wt %, at most 0.6 wt %, at most 0.5 wt %, at most 0.4 wt %, at most 0.3 wt %, or at most 0.2 wt % of the preservative component.

C37. The sunscreen composition of any of paragraphs C0-C36, wherein the sunscreen composition further includes a moisturizing component.

C38. The sunscreen composition of paragraph C37, wherein at least one of the cosmetic emulsion includes the moisturizing component, the moisturizing component is dispersed within the cosmetic emulsion, and the moisturizing component is suspended within the cosmetic emulsion.

C39. The sunscreen composition of any of paragraphs C37-C8, wherein the moisturizing component includes, consists of, or consists essentially of at least one of sodium hyaluronate, sucrose, and D-Panthenol.

C40. The sunscreen composition of any of paragraphs C37-C39, wherein the sunscreen composition includes at least one of:

(i) at least 0.01 wt %, at least 0.05 wt %, at least 0.1 wt %, at least 0.2 wt %, at least 0.3 wt %, at least 0.4 wt %, or at least 0.5 wt % of the moisturizing component; and

(ii) at most 2 wt %, at most 1.8 wt %, at most 1.6 wt %, at most 1.4 wt %, at most 1.2 wt %, at most 1 wt %, at most 0.9 wt %, at most 0.8 wt %, at most 0.7 wt %, at most 0.6 wt %, at most 0.5 wt %, at most 0.4 wt %, at most 0.3 wt %, or at most 0.2 wt % of the moisturizing component.

C41. The sunscreen composition of any of paragraphs C0-C40, wherein the sunscreen composition further includes an anti-foaming component.

C42. The sunscreen composition of paragraph C41, wherein at least one of the cosmetic emulsion includes the anti-foaming component, the anti-foaming component is dispersed within the cosmetic emulsion, and the anti-foaming component is suspended within the cosmetic emulsion.

C43. The sunscreen composition of any of paragraphs C41-C42, wherein the anti-foaming component includes, consists of, or consists essentially of simethicone.

C44. The sunscreen composition of any of paragraphs C37-C39, wherein the sunscreen composition includes at least one of:

(i) at least 0.01 wt %, at least 0.05 wt %, at least 0.1 wt %, at least 0.2 wt %, at least 0.3 wt %, at least 0.4 wt %, or at least 0.5 wt % of the anti-foaming component; and

(ii) at most 1 wt %, at most 0.9 wt %, at most 0.8 wt %, at most 0.7 wt %, at most 0.6 wt %, at most 0.5 wt %, at most 0.4 wt %, at most 0.3 wt %, at most 0.2 wt %, or at most 0.1 wt % of the anti-foaming component.

C45. The sunscreen composition of any of paragraphs C0-C44, wherein the sunscreen composition further includes an anti-inflammatory component.

C46. The sunscreen composition of paragraph C45, wherein at least one of the cosmetic emulsion includes the anti-inflammatory component, the anti-inflammatory component is dispersed within the cosmetic emulsion, and the anti-inflammatory component is suspended within the cosmetic emulsion.

C47. The sunscreen composition of any of paragraphs C45-C46, wherein the anti-inflammatory component includes, consists of, or consists essentially of caffeine.

C48. The sunscreen composition of any of paragraphs C37-C39, wherein the sunscreen composition includes at least one of:

(i) at least 0.01 wt %, at least 0.05 wt %, at least 0.1 wt %, at least 0.2 wt %, at least 0.3 wt %, at least 0.4 wt %, or at least 0.5 wt % of the anti-inflammatory component; and

(ii) at most 1 wt %, at most 0.9 wt %, at most 0.8 wt %, at most 0.7 wt %, at most 0.6 wt %, at most 0.5 wt %, at most 0.4 wt %, at most 0.3 wt %, at most 0.2 wt %, or at most 0.1 wt % of the anti-inflammatory component.

C49. The sunscreen composition of any of paragraphs C0-C48, wherein the sunscreen composition further includes a neutralizing component.

C50. the sunscreen composition of paragraph C49, wherein at least one of the cosmetic emulsion includes the neutralizing component, the neutralizing component is dispersed within the cosmetic emulsion, and the neutralizing component is suspended within the cosmetic emulsion.

C51. The sunscreen composition of any of paragraphs C49-C50, wherein the neutralizing component includes, consists of, or consists essentially of at least one of caprylyl glycol and tromethamine.

C52. The sunscreen composition of any of paragraphs C49-C51, wherein the sunscreen composition includes at least one of:

(i) at least 0.01 wt %, at least 0.05 wt %, at least 0.1 wt %, at least 0.2 wt %, at least 0.4 wt %, at least 0.6 wt %, at least 0.8 wt %, at least 1 wt %, at least 2 wt %, at least 3 wt %, or at least 4 wt % of the neutralizing component; and

(ii) at most 6 wt %, at most 5 wt %, at most 4 wt %, at most 3 wt %, at most 2 wt %, at most 1 wt %, at most 0.8 wt %, at most 0.6 wt %, at most 0.4 wt %, at most 0.2 wt %, or at most 0.1 wt % of the neutralizing component.

C53. The sunscreen composition of any of paragraphs C0-C52, wherein the sunscreen composition further includes a thickening component.

C54. The sunscreen composition of paragraph C53, wherein at least one of the cosmetic emulsion includes the thickening component, the thickening component is dispersed within the cosmetic emulsion, and the thickening component is suspended within the cosmetic emulsion.

C55. The sunscreen composition of any of paragraphs C53-C54, wherein the thickening component includes, consists of, or consists essentially of at least one of disteardimonium hectorite and propylene carbonate.

C56. The sunscreen composition of any of paragraphs C53-C55, wherein the sunscreen composition includes at least one of:

(i) at least 0.001 wt %, at least 0.01 wt %, at least 0.05 wt %, at least 0.1 wt %, at least 0.15 wt %, or at least 0.2 wt % of the thickening component; and

(ii) at most 0.5 wt %, at most 0.4 wt %, at most 0.3 wt %, at most 0.2 wt %, or at most 0.1 wt % of the thickening component.

C57. The sunscreen composition of any of paragraphs C0-C56, wherein the sunscreen composition further includes a filler component.

C58. The sunscreen composition of paragraph C57, wherein at least one of the cosmetic emulsion includes the filler component, the filler component is dispersed within the cosmetic emulsion, and the filler component is suspended within the cosmetic emulsion.

C59. The sunscreen composition of any of paragraphs C57-C58, wherein the filler component includes, consists of, or consists essentially of mica.

C60. The sunscreen composition of any of paragraphs C57-C59, wherein the sunscreen composition includes at least one of:

(i) at least 0.01 wt %, at least 0.05 wt %, at least 0.1 wt %, at least 0.2 wt %, at least 0.4 wt %, at least 0.6 wt %, at least 0.8 wt %, at least 1 wt %, at least 2 wt %, at least 3 wt %, or at least 4 wt % of the filler component; and

(ii) at most 10 wt %, at most 8 wt %, at most 6 wt %, at most 5 wt %, at most 4 wt %, at most 3 wt %, at most 2 wt %, at most 1 wt %, at most 0.8 wt %, at most 0.6 wt %, at most 0.4 wt %, at most 0.2 wt %, or at most 0.1 wt % of the filler component.

C61. The sunscreen composition of any of paragraphs C0-C60, wherein the sunscreen composition further includes a mineral sunscreen component.

C62. The sunscreen composition of paragraph C61, wherein at least one of the cosmetic emulsion includes the mineral sunscreen component, the mineral sunscreen component is dispersed within the cosmetic emulsion, and the mineral sunscreen component is suspended within the cosmetic emulsion.

C63. The sunscreen composition of any of paragraphs C61-C62, wherein the mineral sunscreen component includes, consists of, or consists essentially of at least one of titanium dioxide and zinc oxide.

C64. The sunscreen composition of any of paragraphs C61-C63, wherein the sunscreen composition includes at least one of:

(i) at least 0.001 wt %, at least 0.01 wt %, at least 0.1 wt %, at least 1 wt %, at least 2 wt %, at least 4 wt %, at least 6 wt %, at least 8 wt %, at least 10 wt %, at least 15 wt %, or at least 20 wt % of the mineral sunscreen component; and

(ii) at most 30 wt %, at most 25 wt %, at most 20 wt %, at most 15 wt %, at most 10 wt %, at most 8 wt %, at most 6 wt %, at most 4 wt %, or at most 2 wt % of the mineral sunscreen component.

C65. The sunscreen composition of any of paragraphs C0-C64, wherein the sunscreen composition further includes a surfactant component

C66. The sunscreen composition of paragraph C65, wherein at least one of the cosmetic emulsion includes the surfactant component, the surfactant component is dispersed within the cosmetic emulsion, and the surfactant component is suspended within the cosmetic emulsion.

C67. The sunscreen composition of any of paragraphs C65-C66, wherein the surfactant component includes, consists of, or consists essentially of at least one of an anionic surfactant, a cationic surfactant, an amphoteric surfactant, an ethoxylated surfactant, a lecithin, soy lecithin, Finester 2009, polyglyceryl-3 lactate/laurate, polyglyceryl-3 laurate, and polyglycerin-3.

C68. The sunscreen composition of any of paragraphs C65-C67, wherein the sunscreen composition includes at least one of:

(i) at least 0.001 wt %, at least 0.01 wt %, at least 0.05 wt %, at least 0.1 wt %, at least 0.2 wt %, at least 0.4 wt %, at least 0.6 wt %, at least 0.8 wt %, or at least 1 wt % of the surfactant component; and

(ii) at most 3 wt %, at most 2.5 wt %, at most 2 wt %, at most 1.5 wt %, at most 1 wt %, at most 0.8 wt %, at most 0.6 wt %, at most 0.4 wt %, at most 0.2 wt %, or at most 0.1 wt % of the surfactant component.

D0. A method of forming a sunscreen composition, the method comprising:

providing sunscreen-active material particles that include at least one of:

(i) the mixture of any of paragraphs A0-A32;

(ii) the treated mixture formed via the method of any of paragraphs B0-B20;

(iii) the dewatered treated mixture formed via the method of any of paragraphs B18-B19;

(iv) the chipped treated mixture formed via the method of paragraph B20; and

(v) a dewatered and chipped treated mixture formed via the method of paragraph B20 when dependent from paragraph B19; and

dispersing the sunscreen-active material particles within a cosmetic emulsion.

D1. The method of paragraph D0, wherein the providing includes performing any suitable step and/or steps of any of the methods of any of paragraphs B0-B20.

D2. The method of any of paragraphs D0-D1, wherein the cosmetic emulsion includes any suitable component and/or components of any of the cosmetic emulsions of any of paragraphs C0-C68.

D3. The method of any of paragraphs D0-D2, wherein the dispersing includes:

(i) heating the cosmetic emulsion to a dispersing temperature that is greater than the first glass transition temperature;

(ii) agitating the cosmetic emulsion;

(iii) adding the sunscreen-active material particles to the cosmetic emulsion;

(iv) mixing the sunscreen-active material particles into the cosmetic emulsion to define the sunscreen composition; and

(v) permitting the sunscreen composition to cool.

D4. The method of paragraph D3, wherein a difference between the dispersing temperature and the first glass transition temperature is at least one of:

(i) at least 2° C., at least 4° C., at least 6° C., at least 8° C., at least 10° C., at least 12° C., at least 14° C., at least 16° C., at least 18° C., at least 20° C., or at least 25° C.; and

(ii) at most 30° C., at most 28° C., at most 26° C., at most 24° C., at most 22° C., at most 20° C., at most 18° C., at most 16° C., at most 14° C., at most 12° C., or at most 10° C.

D5. The sunscreen composition formed via any of the methods of any of paragraphs D0-D4 and/or any other steps described herein.

### INDUSTRIAL APPLICABILITY

The sunscreen-active mixtures, sunscreen compositions, and methods disclosed herein are applicable to the sunscreen and cosmetics industries.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

What is claimed is:

1. A sunscreen-active mixture, the mixture comprising:
an inorganic, sunscreen-active material;
a first water-soluble polymer that has a first glass transition temperature of at least 45° C.;
a second water-soluble polymer that has a second glass transition temperature that is less than the first glass transition temperature; and
a surfactant.

2. The mixture of claim 1, wherein the inorganic, sunscreen-active material includes at least one of zinc oxide (ZnO) and titanium dioxide (TiO₂).

3. The mixture of claim 1, wherein the mixture includes at least 1 weight percent (wt %) and at most 90 wt % of the inorganic, sunscreen-active material.

4. The mixture of claim 1, wherein a difference between the first glass transition temperature and the second glass transition temperature is at least 2° C. and at most 30° C.

5. The mixture of claim 1, wherein the first glass transition temperature is at least 35° C. and at most 70° C.

6. The mixture of claim 1, wherein:
(i) the first water-soluble polymer includes at least one of a polyester-5 with a glass transition temperature of approximately 51-55° C., a sulfopolyester with a glass transition temperature of approximately 51-55° C., and a copolymer of diethylene glycol, 1,4-cyclohexanedimethanol and simple esters of isophthalic acid and sulfoisophthalic acid with a glass transition temperature of approximately 51-55° C.; and
(ii) the second water-soluble polymer includes a polyester-5 with a glass transition temperature of approximately 35-38° C., a sulfopolyester with a glass transition temperature of approximately, 35-38° C., and a copolymer of diethylene glycol, 1,4-cyclohexanedimethanol and simple esters of isophthalic acid and sulfoisophthalic acid with a glass transition temperature of approximately 35-38° C.

7. The mixture of claim 1, wherein:
(i) the mixture includes at least 0.001 wt % and at most 60 wt % of the first water-soluble polymer; and
(ii) the mixture includes at least 0.001 wt % and at most 50 wt % of the second water-soluble polymer.

8. The mixture of claim 1, wherein the surfactant includes at least one of an anionic surfactant, a cationic surfactant, an amphoteric surfactant, an ethoxylated surfactant, a lecithin, soy lecithin, polyglyceryl-3 lactate/laurate, polyglyceryl-3 laurate, polyglycerin-3, and polyglycerin-3 (and) polyglyceryl-3 Lactate/Laurate.

9. The mixture of claim 1, wherein the mixture includes at least 0.001 wt % and at most 40 wt % of the surfactant.

10. The mixture of claim 1, wherein the surfactant includes a first surfactant and a second surfactant, wherein the first surfactant includes at least one of a lecithin and soy lecithin, wherein the second surfactant includes at least one of polyglyceryl-3 lactate/laurate, polyglyceryl-3 laurate, polyglycerin-3, and polyglycerin-3 (and) polyglyceryl-3 Lactate/Laurate, and further wherein a mass ratio of the first surfactant to the second surfactant, within the mixture, is 2:3 to 3:2.

11. The mixture of claim 1, wherein the inorganic, sunscreen-active material is a particulate inorganic, sunscreen-active material that include a plurality of material particles, wherein an average particle size of the plurality of material particles is at least 100 nm and at most 15 micrometers.

12. A method of manufacturing a treated mixture for a sunscreen composition, the method comprising:
heating an untreated mixture, which includes the mixture of claim 1, to a heated temperature that is within a threshold temperature differential of the first glass transition temperature of the first water-soluble polymer and greater than the second glass transition temperature of the second water-soluble polymer; and
grinding the untreated mixture to generate the treated mixture.

13. The method of claim 12, wherein the threshold temperature differential is at most at most 5° C.

14. The method of claim 12, wherein the heating includes maintaining the untreated mixture below the first glass transition temperature during the grinding.

15. The method of claim 12, wherein the method further includes measuring an average particle size of the treated mixture subsequent to the grinding.

16. The method of claim 15, further comprising repeating the heating, the grinding, and the measuring the average particle size of the treated mixture until the average particle size of the treated mixture is within a threshold particle size range, wherein the repeating is responsive to the average particle size of the treated mixture, as determined during the measuring the average particle size of the treated mixture, being greater than the threshold particle size range, wherein the threshold particle size range is at least 100 nm and at most 15 micrometers.

17. The method of claim 12, wherein, during the grinding, the method further includes suppressing dust from the untreated mixture, wherein the suppressing includes adding water to the untreated mixture.

18. A sunscreen composition, comprising:
sunscreen-active material particles that include:
(i) an inorganic, sunscreen-active material;
(ii) a first water-soluble polymer that has a first glass transition temperature of at least 45° C.;
(iii) a second water-soluble polymer that has a second glass transition temperature that is less than the first glass transition temperature; and
(iv) a surfactant; and
a cosmetic emulsion;
wherein an average particle size of the sunscreen-active material particles is at least 100 nm and at most 15 micrometers.

19. The sunscreen composition of claim 18, wherein the sunscreen-active material particles include individual sunscreen-active material particles dispersed within the cosmetic emulsion.

20. A method of forming a sunscreen composition, the method comprising:
providing sunscreen-active material particles that include:
(i) an inorganic, sunscreen-active material;
(ii) a first water-soluble polymer that has a first glass transition temperature of at least 45° C.;
(iii) a second water-soluble polymer that has a second glass transition temperature that is less than the first glass transition temperature; and
(iv) a surfactant; and
dispersing the sunscreen-active material particles within a cosmetic emulsion.
